(19) United States
(12) Patent Application Publication
(10) Pub. No.: US 2010/0080785 A1
(43) Pub. Date: Apr. 1, 2010

(54) DRIED FORMULATIONS

(75) Inventor: Volker Berl, New York, NY (US)

Correspondence Address: MORGAN, LEWIS & BOCKIUS LLP (SF), One Market, Spear Street Tower, Suite 2800, San Francisco, CA, 94105, US

(73) Assignee: Zymes, LLC, Hasbrouck Heights, NJ (US)

(21) Appl. No.: 12/420,733

(22) Filed: Apr. 8, 2009

Related U.S. Application Data
(60) Provisional application No. 61/043,345, filed on Apr. 8, 2008.

(51) Int. Cl. A61K 38/43; A61K 31/20

(52) U.S. Cl. 424/94.1; 514/560

(57) ABSTRACT

The present invention provides novel formulations with improved bioavailability for substances such as ubiquinone, ubiquinol and omega-3 fatty acid. The present invention provides methods and compositions for drying formulations of such substances, wherein those dried formulations can readily be dissolved in water. The compositions and methods of the invention are of particular use in adding nutrients, such as vitamins and minerals, to food products such as beverages.

### CROSS-REFERENCE TO RELATED APPLICATIONS

This application claims the benefit under 35 U.S.C. §119(e) of U.S. Provisional Application No. 61/043,345, filed Apr. 8, 2008, which is incorporated by reference in its entirety for all purposes.

### BACKGROUND OF THE INVENTION

Many substances used in food products, cosmetics and pharmaceutical formulations are difficult to incorporate into solutions due to problems with insolubility and pungent taste. The present invention provides methods and compositions for solubilizing such substances.

### SUMMARY OF THE INVENTION

In one aspect, the invention provides a formulation comprising: (a) a lipophilic bioactive molecule; (b) a spray drying carrier; and (c) a solubilizing agent having a structure according to:

Y¹L¹ₐZ

wherein a is an integer selected from 0 and 1; Z is a hydrophobic moiety; Y¹ is a linear or branched hydrophilic moiety including at least one polymeric moiety; and L¹ is a linker moiety that covalently links the hydrophobic moiety Z and the hydrophilic moiety Y¹.

In various embodiments, the lipophilic bioactive molecule is a member selected from a ubiquinone, ubiquinol, carotenoid, xanthophyll, triterpenoid, pentacyclic triterpenoid, phytosterol, stilbenoid (resveratrol), an essential fatty acid, an oil comprising an omega-3 fatty acid, an oil comprising an omega-6 fatty acid, an oil comprising an omega-9 fatty acid and an oil comprising an omega-12 fatty acid. In exemplary embodiments, the lipophilic bioactive molecule is an omega-3 fatty acid or an oil comprising an omega-3 fatty acid. In exemplary embodiments, the lipophilic bioactive molecule is ubiquinone or ubiquinol.

In various embodiments, the solubilizing agent is polyoxyethanyl tocopheryl sebacate (PTS).

In various embodiments, the spray drying carrier comprises a gum and maltodextrin.

In various embodiments, the formulation further comprises a compound selected from the group consisting of a pharmaceutical drug, a sterol, a vitamin, a provitamin, an amino acid, an amino acid analog, a fat, a phospholipid, a carotenoid, a sugar, a starch, an antibiotic, a stabilizer, a reducing agent and a free radical scavenger.

In various embodiments, the weight ratio of the lipophilic bioactive molecule to said solubilizing agent is selected from the group consisting of about 1:2 and about 1:3.

In various embodiments, the weight percent of the bioactive lipophilic molecule is about 1% to about 10%.

In various embodiments, the formulation further comprises water. In various embodiments, the formulation is clear. In various embodiments, the formulation is clear at room temperature. In various embodiments, the formulation is colorless. In one aspect, the invention provides a solid, water-soluble formulation prepared by spray drying any of the preceding formulations.

In one aspect, the invention provides methods of making the formulations disclosed herein. Thus in one aspect, the invention provides a method of making a formulation comprising spray drying a solution comprising said formulation.

### DETAILED DESCRIPTION OF THE INVENTION

### I. Definitions

The term “monoterpene” as used herein, refers to a compound having a 10-carbon skeleton with non-linear branches. A monoterpene refers to a compound with two isoprene units connected in a head-to-end manner. The term “monoterpene” is also intended to include “monoterpenoid”, which refers to a monoterpene-like substance and may be used loosely herein to refer collectively to monoterpenoid derivatives as well as monoterpenoid analogs. Monoterpenoids can therefore include monoterpenes, alcohols, ketones, aldehydes, ethers, acids, hydrocarbons without an oxygen functional group, and so forth.

As used herein, the term “phospholipid” is recognized in the art, and refers to phosphatidyl glycerol, phosphatidyl inositol, phosphatidyl serine, phosphatidyl choline, phosphatidyl ethanolamine, as well as phosphatidic acids, ceramides, cerebrosides, sphingomyelins and cardiolipins.

As used herein, the term “antioxidant” is recognized in the art and refers to synthetic or natural substances that prevent or delay the oxidative deterioration of a compound. Exemplary antioxidants include tocopherols, flavonoids, catechins, superoxide dismutase, lecithin, gamma oryzanol; vitamins, such as vitamins A, C (ascorbic acid) and E and beta-carotene; natural components such as camosol, carnosic acid and rosmanol found in rosemary and hawthorn extract, proanthocyanidins such as those found in grapeseed or pine bark extract, and green tea extract.

The term “flavonoid” as used herein is recognized in the art and is intended to include those plant pigments found in many foods that are thought to help protect the body from cancer. These include, for example, epi-gallo catechin gallate (EGCG), epi-gallo catechin (EGC) and epi-catechin (EC).

The term “alkyl,” by itself or as part of another substituent, means, unless otherwise stated, a straight or branched chain, or cyclic hydrocarbon radical, or combination thereof, which may be fully saturated, mono- or polyunsaturated and can include mono-, di- and multi-valent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)ethyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below as “heteroalkyl,” “cycloalkyl” and “alkylene.” The term “alkylene” by itself or as part of another substituent means a divalent radical derived from an alkane, as exemplified by —CH₂CH₂CH₂CH₂—. Typically, an alkyl group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” refer to those groups having an alkyl group attached to the remainder of the molecule through an oxygen, nitrogen or sulfur atom, respectively. Similarly, the term “dialkylamino” is used in a conventional sense to refer to —NR′R″ wherein the R groups can be the same or different alkyl groups.

The term “acyl” or “alkanoyl” by itself or in combination with another term, means, unless otherwise stated, a stable straight or branched chain, or cyclic hydrocarbon radical, or combinations thereof, consisting of the stated number of carbon atoms and an acyl radical on at least one terminus of the alkane radical.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Also included in the term “heteroalkyl” are those radicals described in more detail below as “heteroalkylene” and “heterocycloalkyl.” The term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini. Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “fluoroalkyl,” are meant to include monofluoroalkyl and polyfluoroalkyl.

The term “aryl,” employed alone or in combination with other terms (e.g., aryloxy, arylthioxy, arylalkyl) means, unless otherwise stated, an aromatic substituent which can be a single ring or multiple rings (up to three rings), which are fused together or linked covalently. “Heteroaryl” are those aryl groups having at least one heteroatom ring member. Typically, the rings each contain from zero to four heteroatoms selected from N, O, and S, wherein the nitrogen and sulfur atoms are optionally oxidized, and the nitrogen atom(s) are optionally quaternized. The “heteroaryl” groups can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl ring systems are selected from the group of acceptable substituents described below. The term “arylalkyl” is meant to include those radicals in which an aryl group is attached to an alkyl group (e.g., benzyl, phenethyl, pyridylmethyl and the like) or a heteroalkyl group (e.g., phenoxymethyl, 2-pyridyloxymethyl, 3-(1-naphthyloxy)propyl, and the like).

Each of the above terms (e.g., “alkyl,” “heteroalkyl” and “aryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from, for example: —OR′, ═O, ═NR′, ═N—OR′, —NR′R″, —SR′, -halogen, —SiR′R″R′″, —OC(O)R′, —C(O)R′, —CO₂R′, CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2N+1), where N is the total number of carbon atoms in such radical. R′, R″ and R′″ each independently refer to hydrogen, unsubstituted (C₁-C₈)alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl, (unsubstituted aryl)-(C₁-C₄)alkyl, (unsubstituted aryl)oxy-(C₁-C₄)alkyl and perfluoro(C₁-C₄)alkyl.

Two of the substituents on adjacent atoms of the aryl ring may optionally be replaced with a substituent of the formula -T-C(O)—(CH₂)_{q}—U—, wherein T and U are independently —NH—, —O—, —CH₂— or a single bond, and the subscript q is an integer of from 0 to 2. Alternatively, two of the substituents on adjacent atoms of the aryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

As used herein, the term “heteroatom” is meant to include, for example, oxygen (O), nitrogen (N), sulfur (S) and silicon (Si).

Certain compounds of the present invention possess asymmetric carbon atoms (optical centers) or double bonds; the racemates, diastereomers, geometric isomers and individual isomers are all encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein, the term “leaving group” refers to a portion of a substrate that is cleaved from the substrate in a reaction. The leaving group is an atom (or a group of atoms) that is displaced as stable species taking with it the bonding electrons. Typically the leaving group is an anion (e.g., Cl⁻) or a neutral molecule (e.g., H₂O). Exemplary leaving groups include a halogen, OC(O)R⁶⁵, OP(O)R⁶⁵R⁶⁶, OS(O)R⁶⁵, and OSO₂R⁶⁵. R⁶⁵ and R⁶⁶ are members independently selected from substituted or unsubstituted alkyl, substituted or unsubstituted aryl, substituted or unsubstituted heteroaryl and substituted or unsubstituted heterocycloalkyl. Useful leaving groups include, but are not limited to, other halides, sulfonic esters, oxonium ions, alkyl perchlorates, sulfonates, e.g., arylsulfonates, ammonioalkanesulfonate esters, and alkylfluorosulfonates, phosphates, carboxylic acid esters, carbonates, ethers, and fluorinated compounds (e.g., triflates, nonaflates, tresylates), SR⁶⁵, (R⁶⁵)₃P⁺, (R⁶⁵)₂S⁺, P(O)N(R⁶⁵)₂(R⁶⁵)₂, P(O)XR⁶⁵X′R⁶⁵ in which each R⁶⁵ is independently selected from the members provided in this paragraph and X and X′ are S or O. The choice of these and other leaving groups appropriate for a particular set of reaction conditions is within the abilities of those of skill in the art (see, for example, March J, ADVANCED ORGANIC CHEMISTRY, 2nd Edition, John Wiley and Sons, 1992; Sandler S R, Karo W, ORGANIC FUNCTIONAL GROUP PREPARATIONS, 2nd Edition, Academic Press, Inc., 1983; and Wade L G, COMPENDIUM OF ORGANIC SYNTHETIC METHODS, John Wiley and Sons, 1980).

“Protecting group,” as used herein refers to a portion of a substrate that is substantially stable under a particular reaction condition, but which is cleaved from the substrate under a different reaction condition. A protecting group can also be selected such that it participates in the direct oxidation of the aromatic ring component of the compounds of the invention. For examples of useful protecting groups, see, for example, Greene et al., PROTECTIVE GROUPS IN ORGANIC SYNTHESIS, 3rd ed., John Wiley & Sons, New York, 1999.

“Ring” as used herein means a substituted or unsubstituted cycloalkyl, substituted or unsubstituted heterocycloalkyl, substituted or unsubstituted aryl, or substituted or unsubstituted heteroaryl. A ring includes fused ring moieties. The number of atoms in a ring is typically defined by the number of members in the ring. For example, a “5- to 7-membered ring” means there are 5 to 7 atoms in the encircling arrangement. The ring optionally included a heteroatom. Thus, the term “5- to 7-membered ring” includes, for example pyridinyl and piperidinyl. The term “ring” further includes a ring system comprising more than one “ring”, wherein each “ring” is independently defined as above.

### II. Introduction

The current invention provides novel formulations with improved bioavailability for substances described herein, including ubiquinone and/or ubiquinol (e.g., CoQ₁₀). In particular, the present invention provides methods and compositions for drying formulations of such substances, wherein those dried formulations can readily be dissolved in water. In an exemplary aspect, the formulations of the invention are spray-dried. Thus, the formulations described herein include dried formulations, liquid formulations and partially dried formulations. In various exemplary embodiments, the liquid formulations are aqueous. The compositions and methods of the invention are of particular use in adding nutrients, such as vitamins and minerals, to food products such as beverages.

### III. Formulations

In one aspect, the invention provides a water-soluble formulation including at least one lipophilic bioactive molecule, an optional water-soluble reducing agent and a solubilizing agent of the invention. Exemplary solubilizing agents are described herein, below. In one example, the solubilizing agent has a structure according to Formula (III) described herein below. In another example, the solubilizing agent has a structure according to Formula (IV), wherein the integer a is selected from 0 and 1:

Y¹L¹ₐZ (IV)

In Formula (IV), Z is a hydrophobic moiety. In one example, Z is a member selected from sterols (e.g., cholesterol or sitosterol), tocopherols (e.g., alpha-tocopherol), tocotrienol and ubiquinols (e.g., ubiquinol-50) and derivatives or homologues thereof. A person of skill in the art will know that the hydrophobic moiety (e.g., tocopherol) when linked to Y¹ is an analog of the molecule, wherein a hydrogen atom is replaced with the moiety “Y¹-[L¹]-”.

In Formula (IV), Y¹ is a linear or branched hydrophilic moiety including at least one polymeric moiety, wherein each polymeric moiety is a member independently selected from poly(alkylene oxides) (e.g., PEG) and polyalcohols. Exemplary lipophilic moieties are described herein, below, each of which is useful in this embodiment. In one example, the lipophilic moiety is poly(ethylene glycol) (PEG) or methylated PEG (mPEG).

In Formula (IV), L¹ is a linker moiety that covalently links the hydrophobic moiety Z and the hydrophilic moiety Y¹. Exemplary linker moieties are described herein below. In one example, L¹ is selected from a single bond, substituted or unsubstituted alkyl, substituted or unsubstituted heteroalkyl, substituted or unsubstituted aryl, substituted or unsubstituted heteroaryl and substituted or unsubstituted heterocycloalkyl. In one embodiment, L¹ includes a linear or branched C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ or C₂₅-C₃₀ alkyl chain, optionally incorporating at least one functional group. Exemplary functional groups according to this embodiment include ether, thioether, ester, carbonamide, sulfonamide, carbonate and urea groups. In a particular example, the solubilizing agent is selected from polyoxyethanyl-tocopheryl-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof.

In an exemplary embodiment, the ratio of the lipophilic bioactive molecule to the solubilizing agent is from about 1:0.3 (w/w) to about 1:20 (w/w). In an exemplary embodiment, the ratio of the lipophilic bioactive molecule to said solubilizing agent is from about 1:1 (w/w) to about 1:20 (w/w). In another exemplary embodiment, the ratio of the lipophilic bioactive molecule to said solubilizing agent is from about 1:1 (w/w) to about 1:10 (w/w). In another exemplary embodiment, the ratio of the lipophilic bioactive molecule to said solubilizing agent is from about 1:1.3 (w/w) to about 1:5 (w/w). In another exemplary embodiment, the ratio of the lipophilic bioactive molecule to said solubilizing agent is from about 1:2 (w/w) to about 1:4 (w/w). In another exemplary embodiment, the ratio of the lipophilic bioactive molecule to said solubilizing agent is about 1:3 (w/w). In an exemplary embodiment, the ratio of the lipophilic bioactive molecule to said solubilizing agent is from about 1:0.3 (w/w) to about 1:1 (w/w). In an exemplary embodiment, the ratio of the lipophilic bioactive molecule to said solubilizing agent is from about 1:0.5 (w/w) to about 1:2 (w/w).

### Water-Soluble Reducing Agent

In an exemplary embodiment, the water-soluble reducing agent contained in the formulation (e.g., aqueous formulation) protects the lipophilic bioactive molecule from chemical degradation (e.g., oxidative and/or light-induced processes). For example, addition of vitamin C or a water-soluble vitamin C derivative to a formulation containing DHA and PTS will serve to prolong the chemical stability of DHA in the aqueous formulation for at least several weeks. In other embodiments, the water-soluble reducing agent is added to the formulation in an amount sufficient to both reduce and stabilize the lipophilic bioactive molecule after reduction. For example, ubiquinone and a solution of a solubilizing agent in water (e.g., PTS) are mixed. Upon mixing of the components, micelles of a small particle size are formed (e.g., average particle size between about 20 and about 30 nm). A water-soluble reducing agent, such as vitamin C or a vitamin C derivative, is then added. The water-soluble reducing agent reduces the ubiquinone to ubiquinol. Excess of water-soluble reducing agent serves to protect against ubiquinol degradation (e.g., oxidation to ubiquinone).

In this function, the water-soluble reducing agent can be considered a stabilizer. In one example, the reducing agent is added in an over-stoichiometric mol ratio with respect to the lipophilic bioactive molecule. In another embodiment, the ratio of lipophilic bioactive molecule to water-soluble reducing agent in the formulation is between about 100:1 and about 1:20 (w/w). In yet another embodiment, the ratio of lipophilic bioactive molecule to water-soluble reducing agent in the formulation is between about 50:1 and about 1:10 (w/w). In yet another embodiment, the ratio of lipophilic bioactive molecule to water-soluble reducing agent in the formulation is between about 20:1 and about 1:10 (w/w). In yet another embodiment, the ratio of lipophilic bioactive molecule to water-soluble reducing agent in the formulation is between about 10:1 and about 1:10 (w/w). In yet another embodiment, the ratio of lipophilic bioactive molecule to water-soluble reducing agent in the formulation is between about 1:1 (w/w) and about 1:10 (w/w), between about 1:1 and about 1:8 (w/w), about 1:1 and about 1:6 (w/w) or between about 1:1 and about 1:4 (w/w). In yet another embodiment, the ratio of lipophilic bioactive molecule to water-soluble reducing agent in the formulation is between about 1:1 and about 1:3 (w/w). In yet another embodiment, the ratio of lipophilic bioactive molecule to water-soluble reducing agent in the formulation is between about 1:1 and about 1:2 (w/w). A person of skill in the art will understand that at least part of the reducing agent can be present in its “oxidized” form. For example, when vitamin C is used as the water-soluble reducing agent, at least part of the vitamin C can be present in the formulation as dehydroascorbic acid. Further details relating to vitamin C, vitamin C derivatives and methods of using these in the present invention can be found in US/2008/0254188 incorporated by reference.

In one example, in which the lipophilic bioactive molecule is an omega-fatty acid (e.g., omega-3-, omega-6- or omega-9-fatty acid), the ratio of fatty acid to water-soluble reducing agent in the formulation is between about 100:1 and about 10:1 (w/w).

In another example, in which the lipophilic bioactive molecule is a carotenoid (e.g., lutein, astaxanthin, canthaxanthin, fucoxanthin or lycopene), the ratio of carotenoid to water-soluble reducing agent in the formulation is between about 10:1 and about 1:10 (w/w).

In one example according to any of the above embodiments, the lipophilic bioactive molecule in the formulation is essentially stable to chemical degradation (e.g., oxidation). In one example, the formulation is essentially stable for at least 30, 60, 90, 120, 160 or 180 days when stored at a temperature below about 25° C. (e.g., about 4° C. or about 10° C.). Typically, the formulations are stored at about 4° C. At this temperature, the formulations are typically stable for at least 4, 5 or 6 month.

In one example, according to any of the above embodiments the formulation is contained in a soft-gelatin capsule. A person of skill will understand that formulations suitable for incorporation into soft-gelatin capsules typically contain less than about 5%, preferably less than about 4%, more preferably less than about 3% and most preferably less than about 2% (w/w) of water. Hence, in one example, the formulation includes less than 5% (w/w) of water.

The lipophilic bioactive molecule in the above formulations can be any lipophilic bioactive molecule, such as those described herein. Exemplary lipophilic bioactive molecules according to any of the above embodiments include those molecules that are difficult to stabilize using known methods. In one example, according to any of the above embodiments, the lipophilic bioactive molecule is a member selected from omega-3-fatty acids (e.g., docosahexaenoic acid (DHA), eicosapentaenoic acid (EPA) and alpha-linolenic acid (ALA)), omega-6-fatty acids, omega-9-fatty acids, carotenoids, essential oils, flavor oils and lipophilic vitamins. Exemplary carotenoids include lutein, astaxanthin, lycopene, fucoxanthin and canthaxanthin. Additional carotenoids (e.g., xanthophylls) are described herein, below.

In one example, according to any of the above embodiments, the formulation is an aqueous formulation and includes at least about 5% (w/w) of water. In other examples, the aqueous formulation includes at least about 10%, at least about 20%, at least about 30% at least about 40% or at least about 50% (w/w) of water. In another example, the aqueous formulation includes more than 50% (w/w) of water. For example, the aqueous formulation includes at least about 55%, at least about 60%, at least about 65%, at least about 70%, at least about 75% or at least about 80% (w/w) of water. In a further example, the aqueous formulation includes more than 80% (w/w) water. For example, the aqueous formulation includes at least about 85%, at least about 90%, at least about 92%, at least about 94% or at least about 96% (w/w) of water.

In one example, the lipophilic bioactive molecule is solubilized in the aqueous formulation through the formation of micelles that are formed between the lipophilic bioactive molecule and the solubilizing agent. The particle size of the formed micelles in solution may be measured using a dynamic light scattering (DLS) detector. Typically, smaller particle sizes are associated with a greater tendency of the human body to absorb active ingredients contained in micelles. In one example, the small size of the micelles, enhances or improves the taste or smell of a flavoring agent. In one embodiment, the aqueous formulations of the invention include micelles with particle sizes smaller than the particle sizes produced by known formulations.

In one embodiment, the aqueous formulation of the invention is essentially clear (e.g., free of precipitation, cloudiness or haziness). In one example, clarity is assessed by the normal human eye. In another example, clarity, haziness or cloudiness of a composition is assessed using light scattering technology, such as dynamic light scattering (DLS), which is useful to measure the sizes of particles, e.g., micelles, contained in a composition or other optical spectroscopic technique. In one example, the lipophilic bioactive molecule of the invention is formulated with PTS resulting in an aqueous formulation that is essentially clear. Clear formulations of the invention can be colored. In one example, the formulation is essentially clear when the micelles have a particle size below a size visible to a human eye (e.g., below 100 nm). Hence, in another exemplary embodiment, the micelles formed between the lipophilic bioactive molecule and the solubilizing agent, have a median (average) particle size of less than about 100 nm. In another example, the micelles formed between the lipophilic bioactive molecule and the solubilizing agent, have a median particle size of less than about 90 nm, less than about 80 nm, less than about 70 nm or less than about 60 nm. In a further example, the micelles formed between the lipophilic bioactive molecule and the solubilizing agent, have a median particle size of less than about 50 nm, less than about 40 nm or less than about 30 nm. In another exemplary embodiment, the average particle size is from about 10 nm to about 90 nm. Another exemplary average particle size is from about 5 nm to about 70 nm, preferably from about 10 nm to about 50 nm, more preferably from about 10 nm to about 30 nm. In a particular example, the micelles formed between the lipophilic bioactive molecule and the solubilizing agent, have a median particle size between about 30 nm and about 20 nm (e.g., about 25 nm).

Alternatively, clarity, haziness or cloudiness of a composition of the invention can be determined by measuring the turbidity of the sample. This is especially useful when the composition is a beverage (e.g., water, soft-drink etc.). In one example, turbidity is measured in FTU (Formazin Turbidity Units) or FNU (Formazin Nephelometric Units). In one example, turbidity is measured using a nephelometer, known in the art. Nephelometric measurements are based on the light-scattering properties of particles. The units of turbidity from a calibrated nephelometer are called Nephelometric Turbidity Units (NTU). In one example, reference standards with known turbidity are used to measure the turbidity of a sample. In one example, a composition of the invention is “essentially clear” when the turbidity is not more than about 500% higher than a control. In another example, a composition of the invention is “essentially clear” when the turbidity is not more than about 300% higher than the control. In yet another example, a composition of the invention is “essentially clear” when the turbidity is not more than about 200%, about 150% or about 100% higher than the control. In a further example, a composition of the invention is “essentially clear” when the turbidity is not more than about 80%, about 60%, about 40%, about 20% or about 10% higher than the control.

The clarity, haziness or cloudiness may be judged relative to the type of formulation considered. In various embodiments, a precursor formulation without solid support as described herein having a nephelometric measurement of less than about 300 NTU, less than about 200 NTU or less than about 100 NTU may be considered clear. In an exemplary embodiment, a precursor formulation without solid support has a nephelometric measurement of less than about 100 NTU and is considered clear. In various embodiments, a powder formulation as described herein dispersed or dissolved in water and having a nephelometric measurement of about 30 NTU to about 150 NTU may be considered clear. In one example, such a formulation is used to deliver about 10 to about 30 mg ubiquinol per serving (e.g., an 8 ounce beverage). In one example, such a formulation is used to deliver about 20 to about 50 mg omega-3 fatty acid per serving (e.g., an 8 ounce beverage).

In another example, the aqueous formulation does not include an alcoholic solvent. For example, the presence of an alcoholic solvent can disrupt the proper formation of the emulsion and can destroy already formed micelles. Exemplary alcoholic solvents that can be detrimental to the micelles formed in aqueous formulations include solvents, such as ethanol, methanol, propanol, butanol and higher alcohols (e.g., C₅-C₂₀ alcohols). Alcoholic solvents also include polyhydric alcohols, such as ethylene glycol, propylene glycol, glycerol and the like. The term “alcoholic solvent” does not include polymers, such as polymeric versions of the above listed polyhydric alcohols (e.g., poly(alkylene oxides)), such as PEG or PPG).

In one example, according to any of the above embodiments, the concentration of lipophilic bioactive molecule in the formulation is at least about 20 mg/mL and can be as high as about 60, about 80, about 100 or more than about 100 mg/mL. In one example, the concentration of lipophilic bioactive molecule in the aqueous formulation of the invention is at least about 1 mg/mL. In another example, the concentration of lipophilic bioactive molecule in the aqueous formulation is at least about 5 mg/mL or at least about 10 mg/mL. In yet another example, the concentration of lipophilic bioactive molecule in the aqueous formulation is at least about 20 mg/mL, at least about 30 mg/mL, at least about 40 mg/mL, at least about 50 mg/mL, at least about 60 mg/mL, at least about 70 mg/mL or at least about 80 mg/mL. In a further example, the concentration of lipophilic bioactive molecule in the aqueous formulation is at least about 85 mg/mL, at least about 90 mg/mL, at least about 95 mg/mL or at least about 100 mg/mL. In yet another example, the concentration of lipophilic bioactive molecule in the aqueous formulation is at least about 110 mg/mL, at least about 120 mg/mL, at least about 130 mg/mL, at least about 140 mg/mL, at least about 150 mg/mL, at least about 160 mg/mL, at least about 170 mg/mL, at least about 180 mg/mL, at least about 190 mg/mL or at least about 200 mg/mL. In another example, the concentration of lipophilic bioactive molecule in the aqueous formulation is greater than 200 mg/mL

In one example, according to any of the above embodiments, the lipophilic bioactive molecule is ubiquinol (e.g., ubiquinol-50) (ubiquinol formulation). Hence, in one embodiment, the invention provides a water-soluble formulation including ubiquinol, a water-soluble reducing agent and a solubilizing agent of the invention. Exemplary solubilizing agents are described herein, below. In one example, the solubilizing agent has a structure according to Formula (IV) described herein.

In one example, the ubiquinol formulation further includes ubiquinone (e.g., CoQ₁₀). In another example, the water-soluble reducing agent used in the ubiquinol formulations is capable of reducing ubiquinone (e.g., CoQ₁₀) to its corresponding ubiquinol (e.g., ubiquinol-50). For example, the formulation is formed by reducing ubiquinone to ubiquinol in situ using a water-soluble reducing agent of the invention (e.g., vitamin C). Such methods are described herein. In one example, this reaction is essentially quantitative. Hence, in another example, the ubiquinol formulation is essentially free of ubiquinone (e.g., CoQ₁₀). Formulations including a small ubiquinone:ubiquinol ratio (e.g., below about 10%) are generally preferred because the reduced version of the molecule is considered the bioactive form and is also more bioavailable than the corresponding ubiquinone. In one example, the ratio of ubiquinone to ubiquinol is less than about 50%, less than about 40%, less than about 30%, less than about 20% or less than about 10% (w/w). In a particular example, the ratio of ubiquinone to ubiquinol in the ubiquinol formulation is less than about 8%, less than about 6%, less than about 4% or less than about 2% (w/w). In another example, the ratio of ubiquinone to ubiquinol in the ubiquinol formulation is less than about 1.8%, less than about 1.6%, less than about 1.4%, less than about 1.2%, or less than about 1% (w/w). In a further example, the ubiquinol formulation is essentially free of ubiquinone (e.g., below HPLC-detectable level). In one example, the ratio of ubiquinol to corresponding ubiquinone is at least about 95%. In another example, the ratio of ubiquinol to ubiquinone is at least about 20, about 40, about 60 or about 80% (w/w).

In a further example according to any of the above embodiments, the ubiquinol formulation contains an amount of the water-soluble reducing agent, which is sufficient to diminish or prevent the chemical degradation of the ubiquinol (e.g., oxidation or re-oxidation to ubiquinone) over time. In this function, the water-soluble reducing agent can be considered a stabilizer. In one example, the reducing agent is added in an over-stoichiometric mol ratio with respect to the ubiquinone/ubiquinol. In another embodiment, the ratio of ubiquinol/ubiquinone to water soluble reducing agent in the ubiquinol formulation is about 1:1 to about 1:50 (w/w). In another embodiment, the ratio of ubiquinol/ubiquinone to water soluble reducing agent in the ubiquinol formulation is about 1:1 to about 1:20 (w/w). In another embodiment, the ratio of ubiquinol/ubiquinone to water soluble reducing agent in the ubiquinol formulation is about 1:1 to about 1:10 (w/w), about 1:1 to about 1:8 (w/w), about 1:1 to about 1:6 (w/w) or about 1:1 to about 1:4 (w/w). In yet another embodiment, the ratio of ubiquinol/ubiquinone to water soluble reducing agent in the ubiquinol formulation is about 1:1 to about 1:3 (w/w). A person of skill in the art will understand that at least part of the reducing agent can be present in its “oxidized” form. For example, when vitamin C is used as the water-soluble reducing agent, at least part of the vitamin C may be present in the ubiquinol formulation as dehydroascorbic acid.

In one example according to any of the above embodiments, the ubiquinol in the ubiquinol formulation is essentially stable to chemical degradation (e.g., oxidation to ubiquinone). In one example, the ubiquinol is essentially stable for at least 30, 60, 90, 120, 160 or 180 days when stored at a temperature below about 25° C. (e.g., about 4° C. or about 10° C.). Typically, ubiquinol formulations are stored at about 4° C. At this temperature, the ubiquinol formulations are stable for at least 90 days. In another embodiment, the aqueous ubiquinol formulation, when stored at about 4° C., is stable for at least 180 days. The extraordinary stability of the reduced form of ubiquinone in the formulations of the current invention constitutes a significant advancement in the art. Such stability is accomplished through a synergistic effect between using an amphiphilic solubilizing agent of the invention, which allows for the formation of unusually small micelles, and the presence of a water-soluble (as opposed to lipid-soluble) reducing agent, such as vitamin C. The discovery that a hydrophobic molecule enclosed in micelles, which expose hydrophilic moieties on their surface, can be effectively reduced by a hydrophilic reducing agent, is surprising.

Another advantage of the above ubiquinol formulations is that they can be essentially colorless. Ubiquinol is much lighter in color (e.g., slight yellow) than the corresponding ubiquinone, which is typically dark orange. The lighter color can be more appealing to the consumer and provides a greater flexibility with respect to the use of coloring agents and other additives. Another advantage of the current formulations stems from the fact that they combine at least two beneficial ingredients (ubiquinone/ubiquinol and vitamin C/vitamin C derivative) in a single preparation. This can provide greater convenience to a consumer. When PTS is used as the solubilizing agent, the instant formulations provide a combination of at least three beneficial ingredients (ubiquinone/ubiquinol, vitamin C/vitamin C derivative and vitamin E) in a single preparation.

In another example according to any of the above embodiments, the ubiquinol formulation is an aqueous formulation. The aqueous formulation can be formed by combining ubiquinone (e.g., CoQ₁₀) with a solution of a solubilizing agent in water forming an emulsion, and subsequently contacting the emulsion with a water-soluble reducing agent to reduce the ubiquinone to ubiquinol. Hence, in another example, the ubiquinol is emulsified in the formulation in the form of micelles that include the ubiquinol and the solubilizing agent. In a typical emulsion of the invention, the micelles are surprisingly small in size. In one example, the micelles are between about 20 and about 30 nm. In another example, the small size of the micelles causes the emulsion to be essentially clear in appearance even at high compound concentrations (e.g., 40, 60, 80 or 100 mg/mL). In one example, the ubiquinol concentration in the aqueous formulations of the invention is at least about 20 mg/mL and can be as high as about 60, about 80, about 100 or more than about 100 mg/mL.

In one example, according to any of the above embodiments, the formulation is water-soluble (water-soluble formulation). In one example, the invention provides a mixture of a water-soluble formulation of the invention and a carrier suitable for topical application. For example, the water-soluble formulation of the invention is used in a skin-care product, such as a cream or ointment.

Beverages

In another example, the invention provides a mixture between a formulation of the invention (e.g., a water-soluble formulation) and an original beverage to create a beverage of the invention. The original beverage can be any beverage (e.g., a clear beverage). Exemplary original beverages are described herein and include carbonated or non-carbonated waters, flavored waters, soft drinks and the like. In one example, the mixture (beverage of the invention) includes between about 1 mg/L and about 1000 mg/L of solubilized lipophilic bioactive molecule. In another example, the mixture includes between about 10 mg/L and about 500 mg/L of solubilized lipophilic bioactive molecule. In yet another example, the mixture includes between about 10 mg/L and about 450 mg/mL, between about 10 mg/L and about 400 mg/mL, between about 10 mg/L and about 350 mg/mL, between about 10 mg/L and about 300 mg/mL, or between about 10 mg/L and about 250 mg/mL of solubilized lipophilic bioactive molecule. In a further example, the mixture includes between about 20 mg/L and about 250 mg/L, between about 20 mg/L and about 200 mg/mL, between about 20 mg/L and about 150 mg/mL, between about 20 mg/L and about 100 mg/mL, or between about 20 mg/L and about 80 mg/mL, between about 20 mg/L and about 60 mg/mL, between about 20 mg/L and about 40 mg/mL of solubilized lipophilic bioactive molecule.

In a particular example according to any of the above embodiments, the invention provides a mixture between a ubiquinol formulation of the invention (e.g., an aqueous ubiquinol formulation) and an original beverage (e.g., carbonated or non-carbonated water) to form a ubiquinol beverage.

Hence, in another aspect, the invention provides a non-alcoholic beverage comprising (a) solubilized ubiquinol (e.g., ubiquinol-50), (b) a water-soluble reducing agent of the invention (e.g., vitamin C) and (c) a solubilizing agent of the invention.

In an exemplary embodiment, the ubiquinol beverage contains between about 1 mg/L and about 1000 mg/L of solubilized ubiquinol. In another example, the beverage contains between about 10 mg/L and about 500 mg/L of solubilized ubiquinol. In yet another example, the mixture includes between about 10 mg/L and about 450 mg/mL, between about 10 mg/L and about 400 mg/mL, between about 10 mg/L and about 350 mg/mL, between about 10 mg/L and about 300 mg/mL, or between about 10 mg/L and about 250 mg/mL of solubilized ubiquinol. In a further example, the mixture includes between about 20 mg/L and about 250 mg/L, between about 20 mg/L and about 200 mg/mL, between about 20 mg/L and about 150 mg/mL, between about 20 mg/L and about 100 mg/mL, or between about 20 mg/L and about 80 mg/mL, between about 20 mg/L and about 60 mg/mL, between about 20 mg/L and about 40 mg/mL of solubilized ubiquinol.

In another aspect, the invention provides a non-alcoholic beverage including (a) solubilized ubiquinone (e.g., CoQ₁₀), (b) a solubilizing agent of the invention, and optionally (c) a water-soluble reducing agent of the invention (e.g., vitamin C).

In one exemplary embodiment, the ubiquinone beverage contains between about 1 mg/L and about 1000 mg/L of solubilized ubiquinone. In another example, the beverage contains between about 10 mg/L and about 500 mg/L of solubilized ubiquinone. In yet another example, the beverage includes between about 10 mg/L and about 450 mg/mL, between about 10 mg/L and about 400 mg/mL, between about 10 mg/L and about 350 mg/mL, between about 10 mg/L and about 300 mg/mL, or between about 10 mg/L and about 250 mg/mL of solubilized ubiquinone. In a further example, the beverage includes between about 20 mg/L and about 250 mg/L, between about 20 mg/L and about 200 mg/mL, between about 20 mg/L and about 150 mg/mL, between about 20 mg/L and about 100 mg/mL, between about 30 and about 100 mg/mL, between about 20 mg/L and about 80 mg/mL, between about 20 mg/L and about 60 mg/mL, or between about 20 mg/L and about 40 mg/mL of solubilized ubiquinone.

In one example according to any of the above aspects, the solubilizing agent has a structure according to Formula (III) described herein below. In another example, the solubilizing agent has a structure according to Formula (IV):

Y¹L¹ₐZ (IV)

wherein the integer a, Y¹, L¹ and Z are defined as herein above. In another example, the solubilizing agent is selected from polyoxyethanyl-tocopheryl-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof.

In one example according to any of the above embodiments, the beverage includes ubiquinol and further includes ubiquinone. In another example according to any of the above embodiments, the beverage includes ubiquinol (e.g., ubiquinol-50) but is essentially free of ubiquinone (e.g., CoQ₁₀).

In a further example according to any of the above embodiments, the beverage further includes a coloring agent and/or a flavoring agent. If required, it is possible to add one or more fruit and/or vegetable juice concentrates and/or flavor improvers to the beverage. For example, a mixture of about LIMETTE citrus (e.g., about 1.38 g/l), cassis (e.g., about 1.04 g/l), mango (e.g., about 1.04 g/l) or combinations thereof, can be added to the beverage. In another example, maltodextrin (e.g., about 20 g/l), fructose (e.g., about 50 g/l) or combinations thereof can be added to the beverage. In another example, the finished beverage is subjected to a primary and, optionally, a secondary filtration. In one example, filters with a pore size of about 0.1μ to about 1.5μ can be used.

In a further example according to any of the above embodiments, the ubiquinol beverage includes sufficient water-soluble reducing agent (e.g., vitamin C) to prevent oxidation of ubiquinol to ubiquinone. In another embodiment, the ratio of ubiquinol/ubiquinone to water soluble reducing agent in the beverage is about 1:1 to about 1:10 (w/w). In another embodiment, the ratio of ubiquinol/ubiquinone to water soluble reducing agent in the beverage is about 1:1 to about 1:8 (w/w), about 1:1 to about 1:6 (w/w) or about 1:1 to about 1:4 (w/w). In yet another embodiment, the ratio of ubiquinol/ubiquinone to water soluble reducing agent in the beverage is about 1:1 to about 1:3 (w/w). A person of skill in the art will understand that at least part of the reducing agent can be present in its “oxidized” form. For example, when vitamin C is used as the water-soluble reducing agent, at least part of the vitamin C can be present in the beverage as dehydroascorbic acid.

In yet another example according to any of the above embodiments, the ubiquinol or ubiquinone is stably solubilized in the beverage. For example, the beverage is essentially free of ubiquinol precipitation and/or ubiquinone precipitation. Hence, in another example, the beverage is essentially clear. Clarity of a beverage can be assessed using turbidity measurements. In one example, the turbidity of the ubiquinol beverage or ubiquinone beverage is comparable (e.g., not more than 5×) of the turbidity of the control beverage. A suitable control is provided by the corresponding original beverage without solubilized ubiquinol/ubiquinone. The control can optionally include the solubilizing agent. In one example, the turbidity of the ubiquinol/ubiquinone beverage is not more than about 500%, not more than about 400%, not more than about 300% or not more than about 200% higher than the turbidity of the control. In yet another example, the turbidity is not more than about 180%, not more than about 160%, not more than about 140%, not more than about 120% or not more than about 100% higher than the turbidity of the control. The turbidity is 100% higher than the control, when the turbidity of the beverage is twice as high as the turbidity of the control. In a further example, the turbidity of the ubiquinol/ubiquinone beverage is not more than about 80%, not more than about 60%, not more than about 40%, not more than about 20% or not more than about 10% higher than the turbidity of the control.

In another example, the turbidity of the ubiquinol/ubiquinone beverage is stable over time. For example, the turbidity of the beverage is stable over a period of at least 60 days when the beverage is stored at ambient temperature (e.g., below about 25° C.).

After production, the beverage can be packaged into opaque containers which are, in particular, opaque to light, such as visible light and near and far ultraviolet light. It is also possible to use for this purpose containers, for example, cans which cover the entire spectrum of light. Cans made of aluminum or aluminum alloys are preferably used. It is also possible to accommodate the beverage according to the invention in metal foil or aluminum foil sachets. In another example, the beverage is packaged in Tetrapak containers. If the material itself does not have the required property of opacity, it can be coated. There is also the possibility of using an opaque outer pack. In one example, the entire production and filling process takes place with essentially exclusion of light.

In addition, the beverage can be vitaminized. In one example, the beverage includes at least one B vitamin. Exemplary B-vitamins include vitamin B1, vitamin B2, vitamin B3 and vitamin B6 and vitamin B12. In another example, the beverage includes vitamin E. In one example, the vitamin is first formulated into an aqueous composition, which is subsequently added to the beverage. The solubilizing agent used to solubilize the vitamin can be the same solubilizing agent used to solubilize the lipophilic bioactive molecule.

### III. (a) Lipophilic Bioactive Molecule

The lipophilic bioactive molecule of the current invention can be any molecule. In one example, the lipophilic bioactive molecule is selected from compounds with a water-solubility that can be increased using a solubilizing agent of the invention. In another example, the bioactive lipophilic molecule is a molecule associated with pharmaceutical or neutraceutical value. The term “lipophilic bioactive molecule” includes derivatives of such molecules (e.g., esters or amides thereof) and combinations thereof. For example, the lipophilic bioactive molecule has at least one free OH or COOH group, which can be converted to an ester group. In another example, the lipophilic bioactive molecule has at least one free primary or secondary amino group, which can be converted to an amide.

### Oils, Fats and Fatty Acids

In an exemplary embodiment, the lipophilic bioactive molecule is an oil or an oil component. The term “oil” includes oils derived from plant material, such as seed oils, essential oils, oils derived from animals, such as fish or marine oils (e.g., salmon oil) and other fats. In one example, the oil has food grade. Exemplary oils derived from plant materials include flaxseed oil, borage seed oil, garlic oil, pumpkin seed oil, evening primrose oil, wheat germ oil, saw palmetto berry oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, jojoba oil and shea butter. Exemplary essential oils include citrus oils, bergamot oil, jasmine oil, ylang ylang oil, rosemary oil, cinnamon oil, lavender oil, rose oil, rose geranium oil, patchouli oil, neroli oil, vetiver oil and the like. The term essential oil also includes fragrances and flavoring oils (e.g., fruit flavor oils, citrus flavor, almond flavor). Exemplary oils derived from animals include animal fats, such as tallow (e.g., beef tallow), butter, chicken fat, lard, dairy butterfat, or combinations thereof.

In another exemplary embodiment, the lipophilic bioactive molecule is selected from an oil comprising at least one fatty acid (e.g., an essential fatty acid). In various embodiments, an oil comprises an omega or omega-n fatty acid, which terms refer to a fatty acid comprising at least one carbon-carbon double bond, with n designating the position of the first carbon-carbon double bond counting from the terminal methyl group. In various exemplary embodiments, n is selected from 3, 6, 9 and 12. In another exemplary embodiment, the lipophilic bioactive molecule is selected from an oil comprising at least one type of an omega-3 fatty acid, an oil comprising at least one type of an omega-6 fatty acid, an oil comprising at least one type of an omega-9 fatty acid and an oil comprising at least one type of an omega-12 fatty acid. Exemplary types of omega-3 fatty acids, omega-6 fatty acids, omega-9 fatty acids and omega-12 fatty acid are disclosed herein below in Table 1.

In various embodiments, the lipophilic bioactive molecule is a fatty acid. In various embodiments, the fatty acid is an omega or omega-n fatty acid. In an exemplary embodiment, the lipophilic bioactive molecule is a member selected from an omega-3 fatty acid, an omega-6 fatty acid, an omega-9 fatty acid, and an omega-12 fatty acid. In an exemplary embodiment, the lipophilic bioactive molecule is an essential fatty acid (EFA), such as a linolenic acid.

In another exemplary embodiment, the lipophilic bioactive molecule is an omega-3 unsaturated fatty acid, such as alpha-linolenic acid (ALA), docosahexaenoic acid (DHA), eicosapentaenoic acid (EPA), stearidonic acid, eicosatetraenoic acid and docosapentaenoic acid. In another exemplary embodiment, the lipophilic bioactive molecule is an omega-6 unsaturated fatty acid, such as linoleic acid, gamma-linolenic acid and arachidonic acid. In yet another exemplary embodiment, the lipophilic bioactive molecule is an omega-9 unsaturated fatty acid, such as oleic acid, eicosenoic acid and erucic acid, as well as conjugated linoleic acid (CLA). In a further exemplary embodiment, the lipophilic bioactive molecule is an omega-12 unsaturated fatty acid. The term “fatty acid” also includes any derivative of those compounds, such as mixed triglycerides, diglyceride esters and alkyl esters, such as methyl- and ethyl esters. Additional fatty acids of the invention are summarized in Table 1, below.

TABLE 1
Exemplary Omega-3, Omega-6 and Omega-9 Fatty Acids

| | | |
|---|---|---|
| | Lipid | |
| Common Name | Name | Chemical Name |

Omega-3 Fatty Acids

| | | |
|---|---|---|
| α-Linolenic acid | 18:3 (n-3) | octadeca-9,12,15-trienoic acid |
| (ALA) | | |
| Stearidonic acid | 18:4 (n-3) | octadeca-6,9,12,15-tetraenoic acid |
| Eicosatetraenoic acid | 20:4 (n-3) | eicosa-8,11,14,17-tetraenoic acid |
| Eicosapentaenoic acid | 20:5 (n-3) | eicosa-5,8,11,14,17-pentaenoic acid |
| (EPA) | | |
| Docosapentaenoic acid | 22:5 (n-3) | docosa-7,10,13,16,19-pentaenoic acid |
| Docosahexaenoic acid | 22:6 (n-3) | docosa-4,7,10,13,16,19-hexaenoic |
| (DHA) | | acid |

Omega-6 Fatty Acids

| | | |
|---|---|---|
| Linoleic acid | 18:2 (n-6) | 9,12-octadecadienoic acid |
| Gamma-linolenic acid | 18:3 (n-6) | 6,9,12-octadecatrienoic acid |
| Eicosadienoic acid | 20:2 (n-6) | 11,14-eicosadienoic acid |
| Dihomo-gamma- | 20:3 (n-6) | 8,11,14-eicosatrienoic acid |
| linolenic acid | | |
| Arachidonic acid | 20:4 (n-6) | 5,8,11,14-eicosatetraenoic acid |
| Docosadienoic acid | 22:2 (n-6) | 13,16-docosadienoic acid |
| Adrenic acid | 22:4 (n-6) | 7,10,13,16-docosatetraenoic acid |
| Docosapentaenoic acid | 22:5 (n-6) | 4,7,10,13,16-docosapentaenoic acid |

Omega-9 Fatty Acids

| | | |
|---|---|---|
| oleic acid | 18:1 (n-9) | 9-octadecenoic acid |
| eicosenoic acid | 20:1 (n-9) | 11-eicosenoic acid |
| mead acid | 20:3 (n-9) | 5,8,11-eicosatrienoic acid |
| erucic acid | 22:1 (n-9) | 13-docosenoic acid |
| nervonic acid | 24:1 (n-9) | 15-tetracosenoic acid |

In another exemplary embodiment, the lipophilic bioactive molecule is a botanical extract or a component thereof. Exemplary extracts include extracts of ginseng, hawthorne, St. John's wort, valerian, black cohosh, yohimbe, ephedra, red clover, cayenne, echinacea, arnica (e.g., arnica montana), grape seeds, kava kava, bilberry, gingko biloba, green tea, wine leaf, Japanese knotwood and any other botanical extract available as a dietary supplement.

In various embodiments, the lipophilic bioactive molecule is a terpene or a terpenoid. The term “terpene” refers to a molecule synthesized by linking isoprene units, which includes activated isoprene units such as isopentenyl pyrophosphate (IPP or also isopentenyl diphosphate), dimethylallyl pyrophosphate (DMAPP or also dimethylallyl diphosphate) and the like. In various embodiments, a terpene has the formula (C₅H₈)—. The term “terpenoid” refers to terpene derivatives, i.e., terpenes that have undergone chemical modifications including but not limited to oxidation and rearrangement. In an exemplary embodiment, the lipophilic bioactive molecule is a carotenoid, such as carotenes and xanthophylls. In one example, the carotene is a member selected from alpha carotene, beta-carotene and lycopene. In another example, the xanthophyll is a member selected from lutein, astaxanthin, zeaxanthin, cryptoxanthin, canthaxanthin, violaxanthin and fucoxanthin.

In another exemplary embodiment, the lipophilic bioactive molecule is a triterpenoid. Triterpenoids include pentacyclic triterpenoids. In one example, the triterpenoid is a member selected from asiatic acid and ursolic acid. In a further exemplary embodiment, the lipophilic bioactive molecule is a sterol or phytosterol. In one example, the phytosterol is a member selected from β-sitosterol and ergosterol. In another exemplary embodiment, the lipophilic bioactive molecule is a stilbenoid. In one example, the stilbenoid is a member selected from resveratrol and pinosylvin.

In an exemplary embodiment, the lipophilic bioactive molecule is a lipophilic vitamin. In an exemplary embodiment, the vitamin is a member selected from vitamin E and vitamin E derivatives. In an exemplary embodiment, the lipophilic bioactive molecule is a member selected from tocopherols and tocotrienols. In another exemplary embodiment, the lipophilic bioactive molecule is a member selected from alpha-tocopherol and alpha-tocotrienol. In another embodiment, the vitamin is a B-vitamin, such as vitamin B pentapalmitate, vitamin B-6 and vitamin B-12.

In yet another exemplary embodiment, the lipophilic bioactive molecule is a member selected from glutathione, catechins, curcumins, lycopene, lecithin, amino acids (e.g., essential amino acids), L-carnitine (or acetyl derivative), alpha-lipoic acid, hyaluronic acid, phytosterols, melatonin and idebenone. In yet another exemplary embodiment, the lipophilic bioactive molecule is a pharmaceutical drug, such as amphotericin B, nystatin, erythromycin, paclitaxel and other anti-tumor agents.

In an exemplary embodiment, the formulation of the invention includes from about 0.01% (w/w) to about 50% (w/w) of a lipophilic bioactive molecule. Formulations including ubiquinol-50, CoQ₁₀ and oils (e.g., DHA oils), typically contain high concentrations of these molecules (e.g., at least 20 mg/mL) as described herein. Formulations including carotenoids (e.g., astaxanthin, fucoxanthin) typically have a lower concentration of these molecules, e.g., due to the fact that they are available only as mixtures (e.g., with oils). A typical carotenoid concentration in the formulation of the invention is between about 1 to 10 mg/mL.

In one example, the formulation includes from about 0.01% (w/w) to about 0.1% (w/w) of a lipophilic bioactive molecule. In another example, the formulation includes from about 0.01% (w/w) to about 0.5% (w/w) of a lipophilic bioactive molecule. In yet another exemplary embodiment, the invention includes from about 0.01% (w/w) to about 1% (w/w) of a lipophilic bioactive molecule. In another exemplary embodiment, the invention includes from about 0.05% (w/w) to about 0.25% (w/w) of a lipophilic bioactive molecule. In a further exemplary embodiment, the invention includes from about 0.1% (w/w) to about 1% (w/w) of a lipophilic bioactive molecule. In another exemplary embodiment, the invention includes from about 0.1% (w/w) to about 0.75% (w/w) of a lipophilic bioactive molecule. In another exemplary embodiment, the formulation includes from about 1% (w/w) to about 3% (w/w) of a lipophilic bioactive molecule. In another exemplary embodiment, the formulation includes from about 1% (w/w) to about 10% (w/w) of a lipophilic bioactive molecule. In another exemplary embodiment, the formulation includes from about 1% (w/w) to about 20% (w/w) of a lipophilic bioactive molecule. In another exemplary embodiment, the formulation includes from about 1% (w/w) to about 30% (w/w) of a lipophilic bioactive molecule. In another exemplary embodiment, the formulation includes from about 1% (w/w) to about 40% (w/w) of a lipophilic bioactive molecule. In another exemplary embodiment, the compositions of the invention contain from about 5% to about 50% by weight of a lipophilic bioactive molecule. In an exemplary embodiment, the composition contains from about 10% to about 30% (w/w) lipophilic bioactive molecule, for example, from about 15% to about 25% (w/w).

### Ubiquinones and Ubiquinols

In an exemplary embodiment, the lipophilic bioactive molecule is an ubiquinone or a reduced form thereof. The reduced form of ubiquinone is generally referred to as an ubiquinol. In an exemplary embodiment, the ubiquinone is ubiquinone Q₁₀ also referred to as coenzyme Q₁₀ (CoQ₁₀). In another exemplary embodiment, the lipophilic bioactive molecule is reduced CoQ₁₀ (ubiquinol-50). In some embodiments, the lipophilic bioactive molecule refers to a composition comprising ubiquinone and ubiquinol in varying ratios as disclosed herein.

In one embodiment, the ubiquinone/ubiquinol of the current invention has a structure according to Formula (I) or Formula (II):

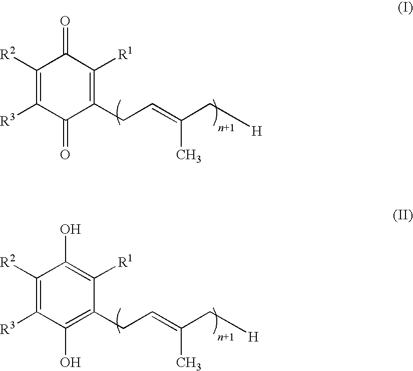

In Formula (I) and Formula (II), the integer n is selected from 1 to 13. R′, R² and R³ are members independently selected from H, substituted or unsubstituted alkyl and substituted or unsubstituted alkoxy. R² and R³, together with the carbon atoms to which they are attached, are optionally joined to form a 5- to 7-membered ring. In one embodiment, n is 9. In another embodiment, R¹ is methyl. In yet another embodiment, R¹ is methyl and R² and R³ are both methoxy. In a preferred embodiment, the ubiquinone of the invention is CoQ₁₀. A preferred ubiquinol is ubiquinol-50, or reduced CoQ₁₀. Also within the scope of the current invention are compositions including both ubiquinone and ubiquinol.

In one example, the compositions of the invention contain from about 5% to about 50% by weight of ubiquinone/ubiquinol. In an exemplary embodiment, the composition contains from about 10% to about 30% (w/w) ubiquinone/ubiquinol, preferably from about 15% to about 25% (w/w). In one embodiment, the soft gelatin capsules of the invention include ubiquinone/ubiquinol from about 1% to about 30% (w/w). In another embodiment the soft gel capsule includes from about 3% to about 20% (w/w), and preferably from about 5% to about 20% of ubiquinone/ubiquinol.

Ubiquinones/ubiquinols can be purchased commercially from sources such as Kaneka (Japan) and Nisshin (Japan). Ubiquinone/ubiquinols can also be synthesized. Exemplary methods are disclosed in U.S. Pat. Nos. 6,545,184 and 6,852,895, U.S. patent application Ser. Nos. 10/992,270; 11/003,544; 11/304,023 and 10/581,566 and U.S. Provisional Patent Application No. 60/804,920, each of which is incorporated herein in its entirety for all purposes.

### III. (b) Solubilizing Agent

In an exemplary embodiment, the solubilizing agent has a structure according to the following formula:

Y¹L¹_{z}Z[L²_{c}Y²]_{b} (III)

In Formula (III), a, b and c are integers independently selected from 0 and 1. In one example, b is 0. Z is a hydrophobic (lipophilic) moiety. In one example, Z is a sterol (e.g., beta-sitosterol, cholesterol). In another example, Z is a tocopherol (e.g., alpha-tocopherol, alpha-tocotrienol) or a derivative or homologue thereof. In yet another example, Z is a ubiquinol. A person of ordinary skill in the art will understand that the residue of the hydrophobic moiety is the entire hydrophobic molecule, except for at least one hydrogen atom, which is replaced with the hydrophilic moiety or the linker-hydrophilic moiety cassette (e.g., hydrogen atom of esterified hydroxyl group, such as 3-β-hydroxyl group of cholesterol or sitosterol or 6-hydroxyl group of α-tocopherol).

In Formula (III), Y¹ and Y² are linear or branched hydrophilic moieties comprising at least one polymeric moiety, wherein each polymeric moiety is independently selected. In one example, Y¹ and Y² are independently selected from hydrophilic (i.e., water-soluble) polymers. In another example, Y¹ and Y² are members independently selected from poly(alkylene oxides) (i.e., polyethers), polyalcohols, polysaccharides (e.g., polysialic acid), polyamino acids (e.g., polyglutamic acid, polylysine), polyphosphoric acids, polyamines and derivatives thereof. Exemplary poly(alkylene oxides) include polyethylene glycol (PEG) and polypropylene glycol (PPG). PEG derivatives include those, in which the terminal hydroxyl group is replaced with another moiety, such as an alkyl group (e.g., methyl, ethyl or propyl). In one example, the hydrophilic moiety is methyl-PEG (mPEG).

PEG is usually a mixture of oligomers characterized by an average molecular weight. In one example, the PEG has an average molecular weight from about 200 to about 5000. In another examplary embodiment, PEG has an average molecular weight from about 500 to about 1500. In another examplary embodiment, PEG has an average molecular weight from about 500 to about 700 or about 900 to about 1200. In one example, the lipophilic moiety of the solubilizing agent is PEG-400. In one example, the lipophilic moiety of the solubilizing agent is PEG-600. Both linear and branched PEG moieties can be used as the hydrophilic moiety of the solubilizing agent in the practice of the invention. In an exemplary embodiment, PEG has between 1000 and 5000 subunits. In an exemplary embodiment, PEG has between 100 and 500 subunits. In an exemplary embodiment, PEG has between 10 and 50 subunits. In an exemplary embodiment, PEG has between 1 and 25 subunits. In an exemplary embodiment, PEG has between 15 and 25 subunits. PEG has between 5 and 100 subunits. In an exemplary embodiment, PEG has between 1 and 500 subunits.

In a further embodiment the poly(ethylene glycol) is a branched PEG having more than one PEG moiety attached. Examples of branched PEGs are described in U.S. Pat. No. 5,932,462; U.S. Pat. No. 5,342,940; U.S. Pat. No. 5,643,575; U.S. Pat. No. 5,919,455; U.S. Pat. No. 6,113,906; U.S. Pat. No. 5,183,660 and WO 02/09766; as well as Kodera Y., Bioconjugate Chemistry 5: 283-288 (1994); and Yamasaki et al., Agric. Biol. Chem., 52: 2125-2127, 1998, all of which are incorporated herein by reference in their entirety. Exemplary branched PEG moieties involve a branched core molecule having at least two PEG arms attached, each at a different attachment point.

In an exemplary embodiment, at least one of Y¹ and Y² includes a moiety having the following structure:

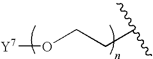

wherein Y⁷ is a member selected CH₃ and H, and n is a member selected from 1 to 5000 (e.g., 1 to 2500). In an exemplary embodiment, n is a member selected from 1000-5000. In an exemplary embodiment, n is a member selected from 1-500. In an exemplary embodiment, n is a member selected from 5-100. In an exemplary embodiment, n is a member selected from 100-500. In an exemplary embodiment, n is a member selected from 10-50. In an exemplary embodiment, n is a member selected from 1-25.

In an exemplary embodiment, Y¹ and/or Y² is a member selected from:

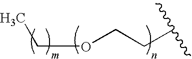

wherein m is a member selected from 0 to 30, and n is a member selected from 1 to 5000. In an exemplary embodiment, m is a member selected from 5-20. In an exemplary embodiment, m is a member selected from 8-15. In an exemplary embodiment, n is a member selected from 1000-5000. In an exemplary embodiment, n is a member selected from 100-500. In an exemplary embodiment, n is a member selected from 10-50. In an exemplary embodiment, n is a member selected from 1-25. In an exemplary embodiment, n is a member selected from 5-100. In an exemplary embodiment, n is a member selected from 1-500.

In an exemplary embodiment, Y¹ and/or Y² is a member selected from:

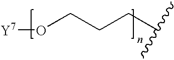

wherein Y⁷ is a member selected CH₃ and H, and n is a member selected from 1 to 2500. In an exemplary embodiment, m is a member selected from 5-20. In an exemplary embodiment, m is a member selected from 8-15. In an exemplary embodiment, n is a member selected from 1000-5000. In an exemplary embodiment, n is a member selected from 100-500. In an exemplary embodiment, n is a member selected from 10-50. In an exemplary embodiment, n is a member selected from 1-25. In an exemplary embodiment, n is a member selected from 5-100. In an exemplary embodiment, n is a member selected from 1-500.

In an exemplary embodiment, Y¹ and/or Y² is a member selected from:

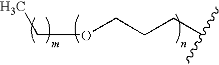

wherein m is a member selected from 0 to 30, and n is a member selected from 1 to 2500. In an exemplary embodiment, m is a member selected from 5-20. In an exemplary embodiment, m is a member selected from 8-15. In an exemplary embodiment, n is a member selected from 1000-5000. In an exemplary embodiment, n is a member selected from 100-500. In an exemplary embodiment, n is a member selected from 10-50. In an exemplary embodiment, n is a member selected from 1-25. In an exemplary embodiment, n is a member selected from 5-100. In an exemplary embodiment, n is a member selected from 1-500.

In one example, the hydrophilic molecule has a reactive functional group, which can be used to chemically attach the hydrophilic molecule to the hydrophobic moiety (e.g., sterol, tocopherol or ubiquinol), either directly or through a linker moiety. Examples of functional groups include esterifiable hydroxyl groups, carboxy groups and amino groups. In one example, the hydrophilic moiety is a polyether (e.g., polyalkylene glycol). The term “polyalkylene glycol” includes polymers of lower alkylene oxides, in particular polymers of ethylene oxide (polyethylene glycols) and propylene oxide (polypropylene glycols), having an esterifiable hydroxyl group at least at one end of the polymer molecule, as well as derivatives of such polymers having esterifiable carboxylic acid groups. The residue of the hydrophilic moiety is the entire hydrophilic molecule, except for the atom involved in forming the bond to the hydrophobic moiety or the linker moiety (i.e. hydrogen atom of an esterified hydroxyl group).

In Formula (III), L¹ and L² are linker moieties. In one example, L¹ and L² are independently selected from a single bond, substituted or unsubstituted alkyl, substituted or unsubstituted heteroalkyl, substituted or unsubstituted aryl, substituted or unsubstituted heteroaryl and substituted or unsubstituted heterocycloalkyl.

In one example, at least one of L¹ and L² includes a linear or branched C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄ or C₂₅-C₃₀ alkyl chain, optionally incorporating at least one functional group. Exemplary functional groups according to this embodiment include ether, thioether, ester, carbonamide, sulfonamide, carbonate and urea groups.

In another example, at least one of L¹ and L² includes a moiety having the following formula:

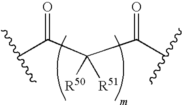

wherein m is an integer selected from 1 to 30. In one example, m is selected from 2 to 20. Each R⁵⁰ and each R⁵¹ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl.

In another example, at least one of L¹ and L² includes a moiety having the following formula:

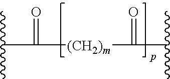

wherein m is an integer selected from 1 to 18 (e.g., from 1 to 10); and p is an integer selected from 0 and 1.

When p is 1, the linker can be derived from an alkanedioic acid of the general formula HOOC—(CH₂)ₘ—COOH. Preferred linkers include diesters derived from an alkanedioic acid. Forr the practice of the present invention, alkanedioic acids with m from 0 to 18 are preferred, those with m from 6 to 10 being particularly preferred. In some embodiments, sebacic acid (m=8) is particularly preferred. In another exemplary embodiment, the solubilizing agent includes the moiety:

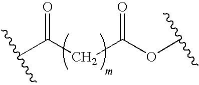

wherein m is a member selected from 4, 6, 8, 10, 12 and 14. In one example, m is 8 and the linker is derived from sebacic acid.

Other preferred linkers include diethers derived from a substituted alkane. In an exemplary embodiment the substituted alkane has the general structure X—(CH₂)—X′ wherein X and X′ independently represent a leaving group such as a halogen atom or a tosylate group. For the practice of the present invention, substituted alkanes with n from 0 to 18 are preferred, those with n from 6 to 10 being particularly preferred. The ether derived from a 1,10-substituted decane (n=10), such as 1,10-dibromodecane is most particularly preferred.

In yet another example, the solubilizing agent includes a moiety, which is a member selected from:

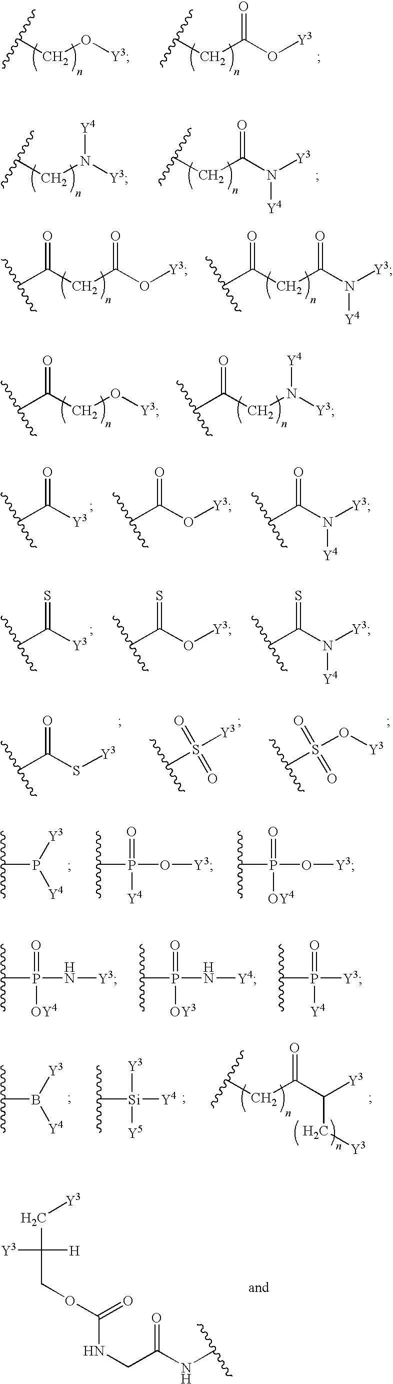

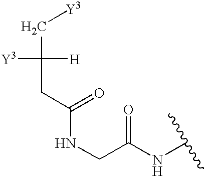

wherein the integer n is a member selected from 0 to 18. Y³ is a member selected from Y¹ and Y². Y⁴ and Y⁵ are members independently selected from H, substituted or unsubstituted alkyl, substituted or unsubstituted heteroalkyl, substituted or unsubstituted aryl, substituted or unsubstituted heteroaryl and substituted or unsubstituted cycloalkyl and substituted or unsubstituted heterocycloalkyl.

In an exemplary embodiment, the solubilizing agent includes a branched linker. In one example, at least one of L¹ and L² includes a moiety having the following formula:

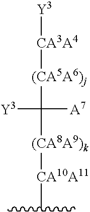

wherein in the integers j and k are independently selected from 0 to 20. A¹, A², A³, A⁴, A⁵, A⁶, A⁷, A⁸, A⁹, A¹⁰ and A¹¹ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, —NA¹²A¹³, —OA¹² and —SiA¹²A¹³. A¹² and A¹³ are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

In one embodiment the solubilizing agent is not PCS (polyoxyethanyl-cholesteryl sebacate). In another embodiment, the solubilizing agent is not TPGS (polyoxyethanyl-a-tocopheryl succinate).

In one exemplary embodiment, the solubilizing agent has a structure according to one of the following formulae:

Y¹-Z-Y²;

Y¹-L¹-Z-Y²;

Y¹-Z-L²-Y²; and

Y¹-L¹-Z-L²-Y²

wherein a, Y¹, Z and L¹ are defined as herein above. All embodiments described herein above for Formula (III) equally apply to the examples of this paragraph.

In one example, the solubilizing agent has a structure according to Formula (IV), wherein the integer a, Y¹, Z and L¹ are defined as herein above:

Y¹L¹ₐZ(IV)

All embodiments described herein above for Formula (III) equally apply to the examples of this paragraph.

### Solubilizing Agents Wherein Z is a Sterol

In an exemplary embodiment, Z is a sterol. In one example, the sterol is a member selected from 7-dehydrocholesterol, campesterol, sitosterol, ergosterol and stigmasterol. Cholesterol and sitosterol are preferred sterols, sitosterol being particularly preferred. In an exemplary embodiment, Z is member selected from a zoosterol and a phytosterol. In another exemplary embodiment, Z is a sterol with an oxygen atom at the 3-position of the A-ring. In an exemplary embodiment, in Formula (IV), Z has a structure according to the following formula:

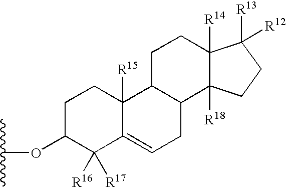

wherein at least one of R¹² and R¹³ is substituted or unsubstituted alkyl. R¹⁴, R¹⁵, R¹⁶, R¹⁷, and R¹⁸ are independently H, or substituted or unsubstituted alkyl. In an exemplary embodiment, Z is a member selected from

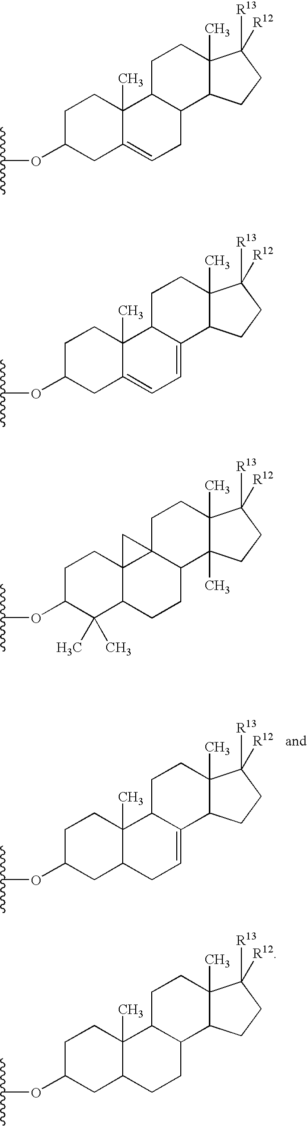

In another example, I the above structures, at least one of R¹² and R¹³ is H. Exemplary sterols according to this embodiment include:

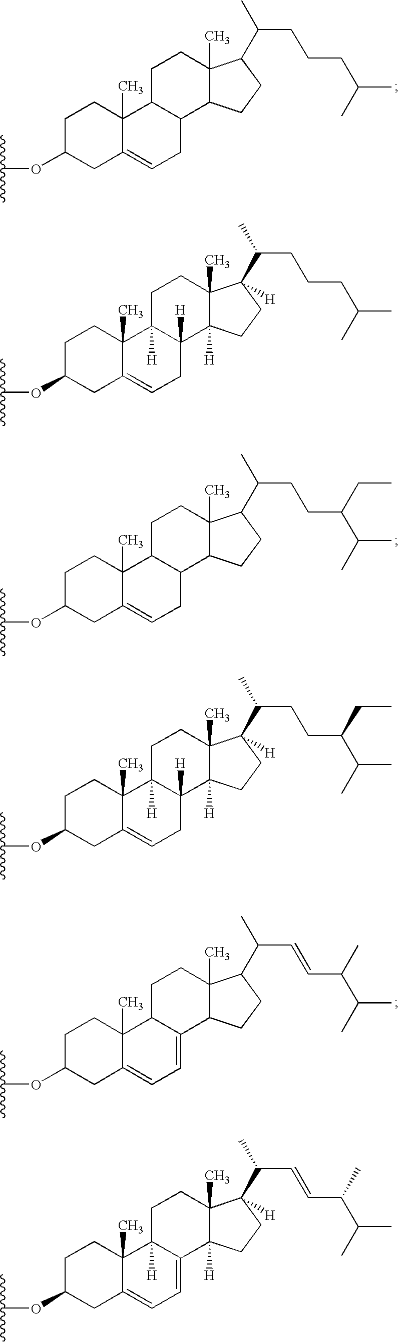

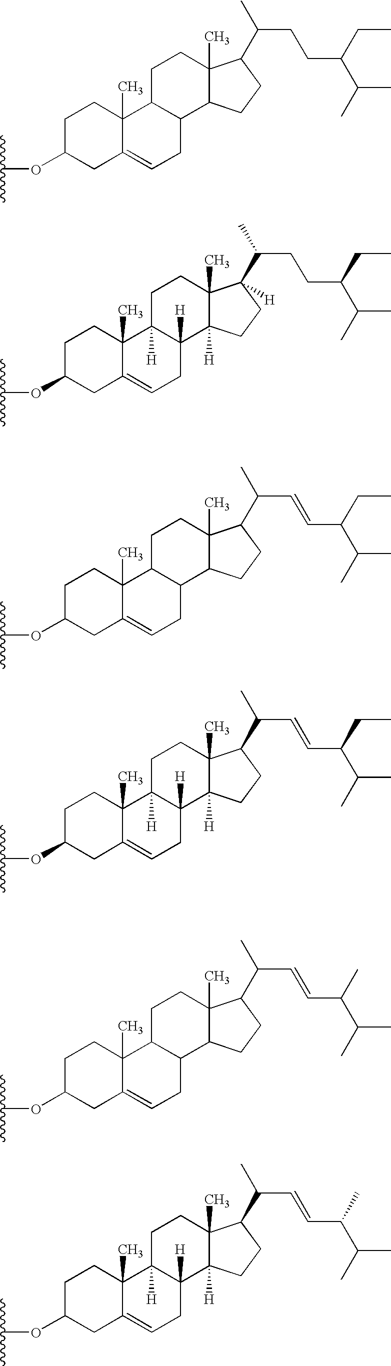

Additional examples of sterols include episterol, cycloartenol, avenasterol, 24-methylenecycloartenol.

Solubilizing Agents wherein Z is a Tocopherol or a Tocotrienol

In another embodiment, Z is a member selected from a substituted or unsubstituted tocopherol and a substituted or unsubstituted tocotrienol. In one example, Z is an α-, β-, γ-, or Δ-tocopherol. α-(+)-Tocopherol and α-(+)-tocopherol are preferred tocopherols, with synthetic DL tocopherol being particularly preferred for PTS. In an exemplary embodiment, Z has a structure according to the following formula:

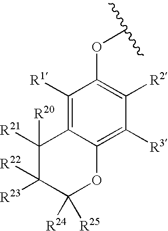

wherein R^{1′}, R^{2′} and R^{3′} are members independently selected from H, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. R^{2′} and R^{3′}, together with the carbon atoms to which they are attached, are optionally joined to form a 5- to 7-membered ring. R²⁰, R²¹, R²², R²³, R²⁴ and R²⁵ are members selected from H, halogen, nitro, cyano, OR¹⁷, SR¹⁷, NR¹⁷R¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. In an exemplary embodiment, at least one of R²⁴ and R²⁵ comprises an isoprene moiety.

In another exemplary embodiment, R^{1′}, R^{2′} and R^{3′} are members independently selected from H and methyl. In another exemplary embodiment, R^{3′} is methyl, R^{2′} is methyl and R^{1′} is methyl. In another exemplary embodiment, R^{3′} is methyl, R^{2′} is H and R^{1′} is methyl. In another exemplary embodiment, R^{3′} is methyl, R^{2′} is methyl and R^{1′} is H. In another exemplary embodiment, R^{3′} is methyl, R^{2′} is H and R^{1′} is H.

In one example, Z has a structure according to the following formulae:

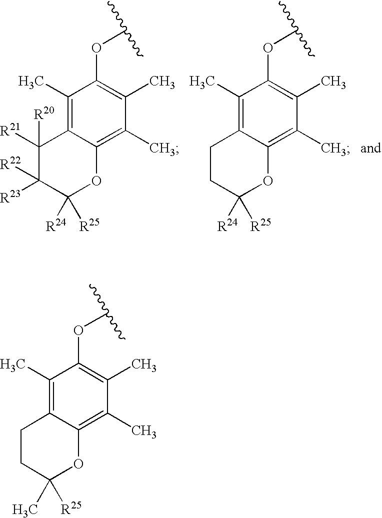

wherein R²⁵ is a member selected from substituted or unsubstituted alkyl and substituted or unsubstituted heteroalkyl. In one example, R²⁴ is methyl. In another example, R²⁵ includes a moiety having a structure selected from the following formulae:

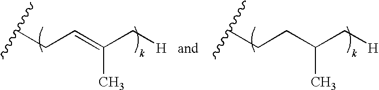

wherein k is an integer selected from 1 to 12. In an exemplary embodiment, k is from 2 to 6. In another exemplary embodiment, k is 3.

In an exemplary embodiment, the solubilizing agent has a structure according to the following formula:

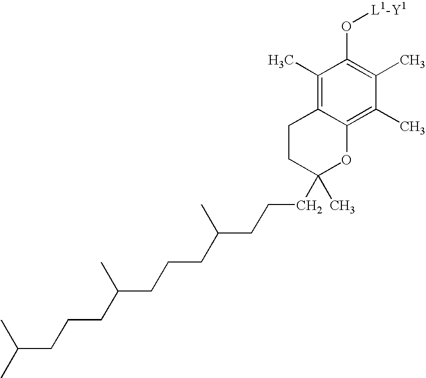

In an exemplary embodiment, the moiety L¹-Y¹ has a structure according to the following formula:

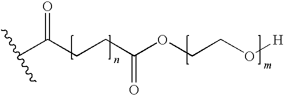

wherein n is member selected from 1 to 20, m is a member selected from 1 to 5000. In another exemplary embodiment, n is 4. In another exemplary embodiment, m is a member selected from 1 to 2,500.

Methods of making the above solubilizing agents are known in the art. For example, the methods of making PCS, PTS, and PSS are disclosed in U.S. Pat. Nos. 6,045,826, 6,191,172, 6,632,443, and WO 96/17626, all herein incorporated by reference. The method of making PQS is disclosed in U.S. Patent Application No. 60/915,061, herein incorporated by reference.

### Specific Sterols and Linkers

In an exemplary embodiment, the solubilizing agent has a structure, which is a member selected from:

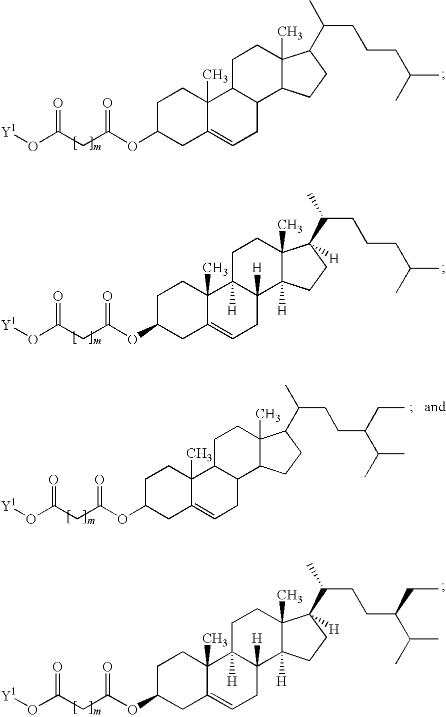

wherein m is a member selected from 2-16. In one example, m is a member selected from 2, 6, 8, 10, 12 and 14. In another example, m is 2. In yet another example, m is 8.

### Specific Sterols and PEG

In an exemplary embodiment, the solubilizing agent is a member selected 0 from

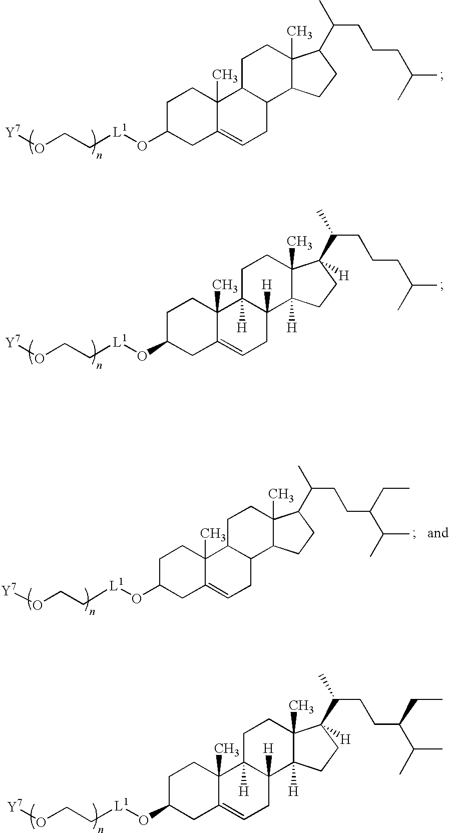

wherein n is a member selected from 10 to 2500, L¹ is a linker moiety, Y⁷ is a member selected from H and methyl.

### Specific Tocopherols and Linkers

In an exemplary embodiment, the solubilizing agent has a structure according to one of the following formulae:

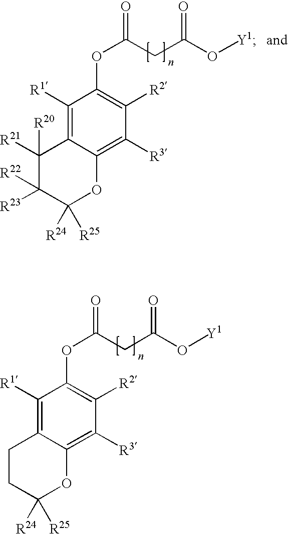

wherein n is an integer selected from 1 to 20. Y¹, R^{1′}, R^{2′}, R^{3′}, R²⁰, R²¹, R²², R²³, R²⁴ and R²⁵ are defined as herein above.

### Specific Tocopherols and PEG

In an exemplary embodiment, the solubilizing agent has a structure according to the following formula:

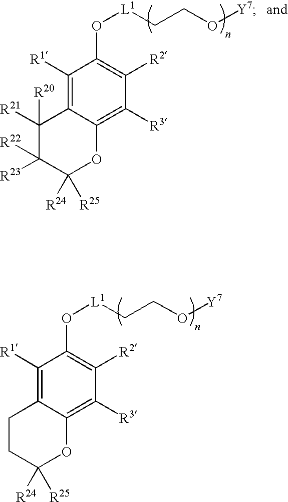

wherein n is a member selected from 10 to 2500. L¹, R^{1′}, R^{2′}, R^{3′}, R²⁰, R²¹, R²², R²³, R²⁴ and R²⁵ are defined as herein above. Y⁷ is selected from H and methyl.

In a preferred embodiment, the solubilizing agent is a member selected from polyoxyethanyl-tocopherol-sebacate (PTS), polyoxyethanyl-sitosterol-sebacate (PSS), polyoxyethanyl-cholesterol-sebacate (PCS), polyoxyethanyl-ubiquinol-sebacate (PQS) and combinations thereof.

In an exemplary embodiment, the formulations of the invention include from about 10% to about 50% by weight of a solubilizing agent, such as PTS. Preferably, the formulations include from about 15% to about 40% (w/w) solubilizing agent, more preferably from about 20% to about 40% (w/w), and even more preferably from about 20 to about 35% (w/w).

In an exemplary embodiment, the invention includes from about 0.01% (w/w) to about 5% (w/w) of a solubilizing agent. In an exemplary embodiment, the invention includes from about 0.01% (w/w) to about 0.1% (w/w) of a solubilizing agent. In an exemplary embodiment, the invention includes from about 0.01% (w/w) to about 1% (w/w) of a solubilizing agent. In an exemplary embodiment, the invention includes from about 0.1% (w/w) to about 1% (w/w) of a solubilizing agent. In an exemplary embodiment, the invention includes from about 0.1% (w/w) to about 0.75% (w/w) of a solubilizing agent. In an exemplary embodiment, the invention includes from about 1% (w/w) to about 3% (w/w) of a solubilizing agent. In an exemplary embodiment, the invention includes from about 0.05% (w/w) to about 0.25% (w/w) of a solubilizing agent.

### IV. Drying Formulations of the Invention

In an exemplary embodiment, the formulations of the invention are dried to a solid form using methods known in the art. Such methods can include without limitation spray drying, nozzle drying (e.g., tower or fountain), wheel drying, flash drying, rotary wheel drying, oven/fluid bed drying, vacuum evaporation, freeze drying, drum drying, tray drying, belt drying, sonic drying, and the like. Although the following discussion provides exemplary embodiments with respect to certain drying techniques, it will be appreciated by one of skill in the art that the present invention is not limited to the following techniques.

### Spray Dried Formulations

In an exemplary aspect, formulations of the invention are spray dried to form a dry powder product. The term “precursor” refers to a formulation that is made before being subjected to a drying technique, resulting in a dry or partially dry solid, such as for example, a dry or partially dry powder. A precursor formulation may include or lack a carrier or solid support as described herein. In one embodiment, a precursor formulation comprises ubiquinol, a solubilizer such as PTS and water, and in an exemplary embodiment, the precursor formulation further comprises a spray drying carrier. In one embodiment, a precursor formulation comprises oil or oil comprising omega-3 fatty acid, a solubilizer such as PTS and water, and in an exemplary embodiment, the precursor formulation further comprises a spray drying carrier. In one example, a water soluble clear formulation of Omega-3 fatty acids or oil comprising omega-3 fatty acids (e.g., Denomega D1000, about 30% in DHA+EPA) (100 g) and PTS (Zymes LLC) (200 g) in water (700 g) is added to a mixture (which can be referred to as a spray drying carrier) of gum and cyclodextrin (400 g). In one example, a formulation is made by combining Omega-3 fatty acids or oil comprising omega-3 fatty acids (e.g., Denomega Omega-Standard, about 30% in DHA+EPA) (100 g), PTS (Zymes LLC) (200 g), water (700 g) and a spray drying carrier comprising a gum and maltodextrin (400 g). In one aspect, the formulation comprising Omega-3 fatty acids or oil comprising omega fatty acids; PTS; spray drying carrier; and water is subjected to regular spray drying conditions known in the art and described herein. The resultant 700 g of white fluffy powder can be collected and readily dispersed in cold or hot water to yield a clear solution.

The terms “solid support”, “spray drying carrier” or “carrier” refers to a substance that facilitates the emulsification, dispersion, dissolution or spray drying of a formulation. Suitable carrier materials include but are not limited to gums (e.g., gum Arabic), starch, gelatin and cellulose derivatives. “Starch” as used herein also includes modified starch or hydrolyzed starch such as, for example, maltodextrin. Other suitable spray drying carriers may be found, for example, in US/2007/0078071 and US/2004/0241444, incorporated by reference in their entirety. In various embodiments, the spray drying carrier comprises a gum; starch; both a gum and starch; any carrier, excipient or additive disclosed herein; and any combination of the foregoing. In various exemplary embodiments, the spray drying carrier comprises a gum and maltodextrin.

In various embodiments, a formulation comprises (a) ubiquinol, (b) a solubilizing agent such as PTS and (c) a spray drying carrier. In various embodiments, a formulation comprises (a) ubiquinol, (b) a solubilizing agent such as PTS, (c) a spray drying carrier and (d) water. In various exemplary embodiments, the spray drying carrier comprises a gum and maltodextrin. In various embodiments, the weight ratio of ubiquinol to PTS is selected from about 1:1, about 1:2, about 1:3, about 1:4, about 1:5 or a range having any of these ratios as endpoints. In various exemplary embodiments, the weight ratio of ubiquinol to PTS is selected from about 1:3, about 1:2 to about 1:3, and about 1:1 to about 1:3. In various exemplary embodiments, the weight ratio of ubiquinol to PTS is about 1:3. In various embodiments, the weight percent of ubiquinol in a formulation is about 1%, about 2%, about 3%, about 4%, about 5%, about 6%, about 7%, about 8%, about 9%, about 10%, or a range having any of these percentages as endpoints. In various exemplary embodiments, the weight percent of ubiquinol in a formulation is selected from at least about 4%, about 2% to about 10%, and about 4% to about 6%. In various exemplary embodiments, the weight percent of ubiquinol in a formulation is at least about 4%. In various exemplary embodiments, the formulation is a dry powder. In various exemplary embodiments, the formulation is a partially dry powder. In various embodiments, the formulation is a liquid formulation.

The dry formulations of ubiquinol disclosed herein showed an increase in stability and shelf life at ambient conditions compared to bulk ubiquinol. At ambient conditions, bulk ubiquinol gradually oxidizes and develops a yellow hue. By contrast, the dry formulations of ubiquinol disclosed herein remains white for weeks at ambient conditions.

In various embodiments, a formulation comprises (a) an omega-n fatty acid or an oil comprising an omega-n fatty acid, (b) a solubilizing agent such as PTS and (c) a spray drying carrier. In various embodiments, a formulation comprises (a) an omega-n fatty acid or an oil comprising an omega-n fatty acid, (b) a solubilizing agent such as PTS, (c) a spray drying carrier and (d) water. In various exemplary embodiments, the spray drying carrier comprises a gum and maltodextrin. In various embodiments, the weight ratio of an omega-n fatty acid or an oil comprising an omega-n fatty acid to PTS is selected from about 1:1, about 1:2, about 1:3, about 1:4, about 1:5, about 1:6, about 1:7, about 1:8 or a range having any of these ratios as endpoints. In various exemplary embodiments, the weight ratio of an omega-n fatty acid or an oil comprising an omega-n fatty acid to PTS is selected from about 1:2, about 1:1 to about 1:2, and about 1:1 to about 1:7. In various exemplary embodiments, the weight ratio of an oil comprising an omega-3 fatty acid to PTS is about 1:2. In various embodiments, the weight percent of an omega-n fatty acid or an oil comprising an omega-n fatty acid in a formulation is about 1%, about 2%, about 3%, about 4%, about 5%, about 6%, about 7%, about 8%, about 9%, about 10%, about 11%, about 12% or a range having any of these percentages as endpoints. In various exemplary embodiments, the weight percent of an omega-n fatty acid or an oil comprising an omega-n fatty acid in a formulation is in a range selected from about 1% to about 10%, about 2% to about 3% and about 7% to about 10%. In various exemplary embodiments, the weight percent of an omega-3 fatty acid in a formulation is about 2% to about 3%. In various exemplary embodiments, the formulation is a dry powder. In various exemplary embodiments, the formulation is a partially dry powder. In various embodiments, the formulation is a liquid formulation. In exemplary embodiments, n is 3.

The dry formulations of omega-3 fatty acid disclosed herein showed an increase in stability and shelf life at ambient conditions compared to bulk omega-3 fatty acid. At ambient conditions, bulk omega-3 fatty acid gradually oxidizes and develops a fishy smell. By contrast, the dry formulations of omega-3 fatty acid disclosed herein remains odorless for weeks at ambient conditions.

In a general aspect, spray drying begins with the atomization of a liquid feedstock into a spray of droplets. The droplets are then put into contact with hot air in a drying chamber. The sprays are produced by either rotary (wheel) or nozzle atomizers, which are widely available and known in the art. Evaporation of moisture from the droplets and formation dry particles proceed under controlled temperature and airflow conditions, and powder is continuously discharged from the drying chamber and recovered from the exhaust gases using a cyclone or a bag filter. The recovery can take place within seconds.

A typical spray dryer consists of a feed pump, atomizer, air heater, air disperser, drying chamber, systems for powder recovery and exhaust air cleaning, with the appropriate process control systems.

Spray drying is a well known process long used, e.g., in the food processing industry to produce powders. For example, liquid products, such as milk, are sprayed through a nozzle into a stream of hot gasses to produce a powder. The increased surface area exposed in the spray mist, in combination with the high temperatures of the drying gas, provides rapid removal of water from the liquid product.

The suspension or solution of the invention is, e.g., mixed in a chamber with a high-pressure gas or a near supercritical fluid before spraying through a capillary restrictor nozzle outlet to form a fine mist of droplets. Without being bound to a particular theory, the combination of a high pressure gas or a near supercritical fluid with the suspension or solution can provide an emulsion mixture of droplets saturated and/or surrounded with fluid under pressure. As the mixture is released from the spray nozzle, the pressure drops rapidly allowing an explosive expansion, and/or effervescence (degassing), that disrupts the droplets into a fine mist (gaseous suspension of droplets). Such a mist can be, e.g., finer than would result with spraying at a lower pressure (e.g. less than 100 psi) or spraying without a near supercritical fluid. The droplets can experience, e.g., cool temperatures during any phase transition or adiabatic expansion associated with the decompression of the mixture. Shear stress can less than with hydraulic spraying (i.e., spraying liquid without gas) at a pressure high enough to provide the same fine droplets.

The suspensions or solutions are combined with a near supercritical fluid and/or high-pressure gas, e.g., in a mixing chamber before spraying to expand in a particle formation chamber. The suspension or solution can be held in a container (first chamber) and supplied through a conduit to the mixing chamber. The suspension or solution can be forced into the mixing chamber, e.g., by pressurization of the container or by pumping through high pressure pump. The high-pressure gas and/or near supercritical fluid can be supplied to the mixing chamber, e.g., through a conduit from a pressurized vessel (second chamber). The mixing chamber can be, e.g., an expanded conduit within the nozzle structure configured to produce vortices or turbulence in the flowing mixture. Depending, for example, on the gas or fluid, and the suspension or solution constituents, the bioactive material can exist as a particle, emulsion, precipitate, and/or solute in the mixture.

The spray nozzle of the invention can be adapted to provide the desired fine mist of droplets. The nozzle can have, e.g., a conduit feeding the mixture to a capillary restrictor spray orifice that has an internal diameter of between about 50 μm and about 1000 μm, or about 100 μm. In a preferred embodiment, the mixture comprises an emulsion of the suspension or solution in the pressurized gas or near supercritical fluid, such that when the pressure is rapidly reduced, the fluid rapidly transitions to gas, dispersing the emulsion droplets. The pressure release can be, e.g., rapid enough that the gas formation is explosive, causing the formation of fine droplets comprising the bioactive material. More specifically, it has been found that supercritical CO₂ assisted spraying results in the generation of ultra fine spray droplets.

As will be appreciated by one of skill in the art, control of parameters such as particle size, size distribution, shape and form in the particulate product will be dependent upon the operating conditions used when carrying out the methods of the invention. Variables include the flow rate of the supercritical fluid, flow rate of the solution or suspension, the concentrations of the bioactive material and excipients, diameter and length of the nozzle, the surface charge on the particles, and the relative humidity, temperature, and pressure inside the particle formation chamber and secondary drying chamber.

The flow rates of the high-pressure gas/near supercritical fluid and/or the suspension/solution through the nozzle can be controlled to achieve a desired particle size, size distribution, shape, and/or form. The flow rates can be established by adjusting independent valves in the conduits, which are preferably needle valves. Flow rates can also be controlled by altering pumping conditions for the high-pressure gas/near supercritical fluid and/or the suspension/solution. Droplets in the invention are typically produced with an average size ranging from about 1 μm to about 50 μm, or about 5 μm, before drying into particles.

Near supercritical fluid is typically introduced into the mixing chamber at a near the critical pressure of the fluid. High-pressure gas is typically introduced into the mixing chamber at pressures above about 1000 psi. The suspension or solution is typically introduced into the mixing chamber at a flow rate from about 0.5 ml/min to about 50 ml/min, or about 3 ml/min (for a 100 um capillary restrictor) to about 30 ml/min, and at a pressure near the pressure of the supercritical fluid. The mass flow ratio (gas/liquid) of the high-pressure gas or near supercritical fluid flow rate to the suspension or solution flow rate can be between about 0.1 and 100, preferably between 1 and 20, more preferably between 1 and 10, and most preferably around 5. Higher proportions and higher flow rates of suspension or solution can increase the size of the droplets and the dry particles. Dry powder particles in the invention can be controlled to have an average diameter, e.g., less than about 200 μm, from about 0.5 μm to about 150 μm, typically from about 1 um to about 15 μm; preferably, from about 3 μm to about 10 μm; and most preferably, from about 5 μm to about 10 μm. Droplet sizes (measured as the mass median diameter—MMD) can be controlled to have a range from about 1 μm to 400 μm, from about 1 μm to about 200 μm; preferably from about 5 μm to about 50 μm; and most preferably from about 3 μm to about 10 μm.

Pressurized gases that are suitable for spraying solutions or suspensions of the invention include, e.g. nitrogen, carbon dioxide, oxygen, propane, nitrous oxide, helium, hydrogen, and/or the like; at pressures ranging from about 100 pounds per square inch (psi) to about 15,000 psi. A number of fluids suitable for use as supercritical fluids are known to the art, including, e.g., carbon dioxide, sulfur hexafluoride, chlorofluorocarbons, fluorocarbons, nitrous oxide, xenon, propane, n-pentane, ethanol, nitrogen, water, other fluids known to the art, and mixtures thereof. The supercritical fluid is preferably carbon dioxide or mixtures of carbon dioxide with another gas such as fluoroform, and/or modifiers, such as ethanol. The temperature of pressurized gases and/or supercritical fluids mixed with suspensions or solutions in the methods can be, e.g., from about 0° C. to about 60° C. In a typical embodiment, the near supercritical fluid is CO₂ at a pressure of about 1000 psi. Fine particles can also be dispersed under lower carbon dioxide pressures, e.g., 500, 750 and 950, (under near-critical conditions). Near-critical fluids are defined (King, M. B., and Bott, T. R., eds. (1993), “Extraction of Natural Products using Near-Critical solvents,” (Blackie Acad & Prof., Glasgow) pp. 1-33) as substances maintained at pressures between 0.9 and 1.0 of their critical pressure and/or temperature (in degree Kelvin).

The supercritical fluid can optionally contain one or more modifiers, for example, but not limited to, methanol, ethanol, isopropanol, and/or acetone. When used, the modifier preferably constitutes not more than 20%, and more preferably constitutes between 1 and 10%, of the volume of the supercritical fluid. The term “modifier” is well known to those persons skilled in the art. A modifier (or co-solvent) may be described as a chemical which, when added to a supercritical fluid, changes the intrinsic or colligative properties of the supercritical fluid in or around its critical point.

Primary drying of the droplets can begin, e.g., during the expansion of the gas-liquid mixture. Primary drying can, e.g., convert liquid droplets into primarily dried particles. Some of the solvent of the suspension or solution can be dissolved in the near supercritical fluid, e.g., even before the expansion begins. As the spray expands, the fluid can change state to a gas, removing latent heat and cooling the mist. The explosive expansion can break mixed droplets into smaller droplets. Degassing of high-pressure gases or supercritical fluids out of the droplets can further disrupt them into finer droplets. The gasses and vapors around the fine droplets can be displaced by (i.e., be exchanged with) a stream of drying gas flowing through the particle formation vessel. Significant amounts of solvent can be evaporated from the fine droplets on contact with the drying gasses; this can be accelerated by the high surface to volume ratio of the droplets, a warm temperature of the drying gas, and a low relative humidity of the drying gas. Secondary drying can take place in the particle formation vessel and/or the drying gas can carry the fine droplets and/or primarily dried particles to a secondary drying chamber for further reduction of residual moisture.

Optionally, the fine mist of droplets can be sprayed into a stream of cold fluid to freeze the droplets. The cold stream can be, e.g., a gas (e.g., CO₂), or a liquid (liquid nitrogen), at temperature between about −60° C. to about −200° C. The frozen droplets can be exposed to an environment of low pressure (i.e., a pressure less than atmospheric) to remove ice by sublimation to form, e.g., low density, lyophilized dry powder particles.

Secondary Drying

Secondary drying of the structurally stabilized and primarily dried particles can, e.g., further remove entrapped solvent, residual moisture, and/or water of molecular hydration, to provide a composition of powder particles with significantly lower moisture content that is stable in storage, e.g., for extended periods at ambient temperatures. Secondary drying can involve, e.g., suspension of particles in a vortex of drying gas, suspension of particles in a fluidized bed of drying gas, and/or application of warm temperatures to the particles in a strong vacuum for several hours to days. The rapid drying and fine particle sizes formed during spraying and primary drying can allow reduced temperatures and times for secondary drying in methods of the invention.

During the secondary drying process, e.g., a spray coat or other protective coating can be applied to the particles. For example, a mist of a polymer solution can be sprayed into a suspension of drying particles in a vortex or fluidized bed.

The methods of the invention result in a pharmaceutically-acceptable, powder particles. In an exemplary embodiment, the composition is almost completely dry. Some water or other aqueous solvent can remain in the composition but typically, not more than about 5% residual moisture remains by weight. The drying temperature can range from less than about 90° C., between about 25° C. and about 80° C., between about 30° C. and about 50° C. or about 35° C. A typical secondary drying process can include, e.g., raising the temperature to a drying temperature of from about 30° C. to about 55° C., and holding for from about 0.5 days to about 5 days to provide a stable dried powder composition with 0.1% to about 5%, or about 3% residual moisture. As used herein, “dry”, “dried”, and “substantially dried” encompass those compositions with from about 0% to about 5% water. Preferably, the powder matrix will have a moisture content from about 0.1% to about 3%.

Apparatus of the Invention

The apparatus of the invention can include, e.g., a container (first chamber) to hold the suspension or solution, a pressure vessel (second chamber) to hold a high-pressure gas and/or near supercritical fluid, conduits with control valves to control flow from the first and second chambers into a mixing chamber, a nozzle with a capillary restrictor through which a mixture can be sprayed into a particle formation vessel, and a flow of drying gas that can provide primary and/or secondary drying of particles from the particle formation vessel. Secondary drying of particles can include, e.g., settling to a warm surface in a vacuum, lyophilization of frozen particles, suspension in a vortex of drying gas, and/or suspension in a fluidized bed of drying gas.

Certain chambers and vessels of the apparatus can have multiple or alternate functions to carry out the methods of the invention. For example, in some embodiments, the particle formation vessel can also act as a secondary drying chamber, and/or a particle collection vessel. Optionally, the secondary drying chamber can comprise a vortex chamber, fluidized bed chamber, a particle sizing chamber, a polymer coating chamber, and/or a particle collection vessel.

Fluids and Gasses

The apparatus of the invention can have chambers and conduit to hold and transfer the high-pressure gas or near supercritical fluids, and suspensions or solutions, to a mixing chamber. The sprayed droplets can experience primary and secondary drying, e.g., by contact with drying gases.

The high-pressure gases and/or near supercritical fluids can include without limitation nitrogen, carbon dioxide, sulfur hexafluoride, chlorofluorocarbons, fluorocarbons, nitrous oxide, xenon, propane, n-pentane, ethanol, nitrogen, water, and/or the like. Modifiers, such as certain alcohols can be dissolved in the supercritical fluids to, e.g., adjust the solvent, critical point and/or expansion properties of the fluid.

The suspensions or solutions of the apparatus can include additional excipients, such as surfactants and carriers as described herein.

Apparatus Hardware

The apparatus of the invention can include, e.g., a first chamber to hold a suspension or solution, a second chamber to hold a high-pressure gas and/or near supercritical fluid, a nozzle with a mixing chamber and a capillary restrictor with an outlet orifice, a particle formation chamber, and a secondary drying chamber. Suspension or solution can be pumped into the mixing chamber under pressure through a first conduit to mix with near supercritical fluid pumped into the mixing chamber through a second conduit. The mixture can spray out of the nozzle as a mist into the particle formation chamber where it can begin to dry on contact with a stream of drying gas. Secondary drying can take place by contact with warmed chamber walls and/or by contact with the stream of drying gas in the particle formation vessel and/or a secondary drying chamber.

In a preferred embodiment, the particle formation vessel and/or secondary drying chamber are housed within an environmental control chamber. The controlled humidity and temperature of the environmental control chamber can be the source of drying gases. Inlet gas from the environmental control chamber to the particle formation vessel can be mixed with droplets emitted from the capillary constrictor as a fine mist. The fine mist can be partially dried (i.e., from a droplet into a particle) in the particle formation vessel before transfer in a stream of drying gas to a secondary drying chamber, such as a cyclonic vortex chamber. The stream of drying gas can continue to a gas outlet port back into a environmental control chamber where the gas can be reconditioned. The apparatus can further comprise a desiccant or condenser system for removing moisture from the gas and/or the environmental control chamber. A heat exchanger can be used to control the temperature of the recycled gas and prevent excessive build up of temperature inside the environment controlled chamber. Typically, the chamber is cooled by introduction of liquid nitrogen from a liquid nitrogen reservoir with control by an optional temperature controller which can automatically meter the liquid nitrogen to provide for a relatively invariant temperature inside the environmental control chamber. Optionally, the environmental control chamber can be cooled by a refrigeration heat exchanger (evaporator). The environmental control chamber is typically vented to the ambient room pressure via a pressure control port which can be valved or pressure gated. Spray drying into a reduced moisture controlled gas can provide a large moisture differential between the sprayed droplets and the drying chamber environment. The effect can be a reduced input heat requirement for the primary drying phase.

The first and second chambers can be pressurized, and/or pumps can be employed in the conduits, to deliver high-pressure gas and/or near supercritical fluid, and/or suspensions or solutions, to the mixing chamber. The rate of delivery can be controlled by means commonly practiced in the art, such as, e.g., by controlling the pumping rate or by controlling valves in the conduits. The pumps can be any type known in the art, such as, e.g., peristaltic pumps, rotary pumps, diaphragm pumps, piston pumps, and the like. Valves can be any appropriate style known in the art, including, e.g., ball and seat, diaphragm, needle, that can restrict the flow of pressurized fluids. Typically, the second container is pressurized, refrigerated and/or insulated to hold the pressurized gas or fluid at near critical conditions.

The mixture sprays out of the nozzle into a particle formation vessel where it, e.g., expands to gases and disrupted fluid feed droplets. A drying gas can be introduced into the particle formation vessel to displace mixture gasses (expanded gases and evaporated solvents) from the droplets. The drying gasses can contact the droplets to evaporate additional solvent from them to form particles. The drying gasses can carry droplets and/or particles to other chambers for processing by the methods of the invention. For example, primarily dry particles can be suspended in a stream of drying gas in the particle formation vessel, or be carried to a separate chamber, for secondary drying, sizing, coating, and/or collection. The drying gas can be, e.g., an inert gas, such as nitrogen, at a temperature below the glass transition temperature of the powder particles. The apparatus can include heat exchangers to control the temperature of the drying gas, e.g., less than about 90° C., between about 25° C. to about 80° C., between about 30° C. and 50° C., or about 35° C. Preferred drying gas (inlet gas) temperatures during particle formation in the methods of the invention are less than 65° C., or between about 30° C. and about 55° C., or about 35° C. The apparatus can include condensers or desiccators to lower the relative humidity, or solvent level, of the drying gas, e.g., so it can be recycled or sent to waste without harm to the environment.

### Drum-Dried Formulations

One of the most economical drying methods is drum drying. In this operation, food slurry is contacted with a hot, revolving drum to form a thin layer on the surface. After sufficient residence time to allow the evaporation of water the product is removed from the drum by a scraper device (called a doctor knife) located usually ½ to ¾ of a revolution from the point of application.

Factors affecting the rate of drying and final moisture content are: residence time on the drum; surface temperature; film thickness. The method of dehydration can be applied to food slurries or liquid food systems and the product must be able to withstand high temperature-short time exposures without undergoing severe quality changes.

### Freeze-Dried Formulations

Freeze drying (also known as lyophilization) is a dehydration process typically used to preserve a perishable material or make the material more convenient for transport. Freeze drying works by freezing the material and then reducing the surrounding pressure and adding enough heat to allow the frozen water in the material to sublime directly from the solid phase to gas. There are three stages in the complete freeze-drying process: Freezing, Primary Drying, and Secondary Drying.

The Freezing process consists of freezing the material. This can be done by placing the material in a freeze-drying flask and rotating the flask in a bath, called a shell freezer, which is cooled by mechanical refrigeration, dry ice and methanol, or liquid nitrogen. On a larger-scale, freezing can done using a freeze-drying machine. In this step, it is important to cool the material below its eutectic point, the lowest temperature at which where the solid and liquid phase of the material can coexist. This ensures that sublimation rather than melting will occur in the following steps. Larger crystals are easier to freeze dry. To produce larger crystals the product should be frozen slowly or can be cycled up and down in temperature. This cycling process is called annealing. However, in the case of food, or objects with formerly living cells, large ice crystals will break the cell walls. Generally, the freezing temperatures are between −50° C. and −80° C.

During the Primary Drying phase the pressure is lowered (to the range of a few millibar) and enough heat is supplied to the material for the water to sublimate. The amount of heat necessary can be calculated using the sublimating molecules' latent heat of sublimation. In this initial drying phase about 95% of the water in the material is sublimated.

In this phase, pressure can be controlled through the application of partial vacuum. The vacuum speeds sublimation making it useful as a deliberate drying process. Furthermore, a cold condenser chamber and/or condenser plates provide a surface(s) for the water vapour to re-solidify on. This condenser plays no role in keeping the material frozen; rather, it prevents water vapor from reaching the vacuum pump, which could degrade the pump's performance. Condenser temperatures are typically below ±50° C. (−58° F.). It is important to note that in this range of pressure, the heat is mainly brought by conduction or radiation, the convection effect can be considered as insignificant.

The Secondary Drying phase aims to sublimate the water molecules that are adsorbed during the freezing process, since the mobile water molecules were sublimated in the primary drying phase. This part of the freeze-drying process is governed by the material's adsorption isotherms. In this phase, the temperature is raised higher than in the primary drying phase, and can even be above 0° C., to break any physico-chemical interactions that have formed between the water molecules and the frozen material. Usually the pressure is also lowered in this stage to encourage sublimation (typically in the range of μbar). However, in some embodiment, increased pressure may be applied.

After the freeze drying process is complete, the vacuum is usually broken with an inert gas, such as nitrogen, before the material is sealed. At the end of the operation, the final residual humidity in the product is generally around 1% to 4%.

### V. Forms of Administration of the Formulations

The formulations of the invention can take a variety of forms adapted to the chosen route of administration. Those skilled in the art will recognize various synthetic methodologies that may be employed to prepare non-toxic pharmaceutical formulations incorporating the compounds described herein. Those skilled in the art will recognize a wide variety of non-toxic pharmaceutically acceptable solvents that may be used to prepare solvates of the compounds of the invention, such as water, ethanol, propylene glycol, mineral oil, vegetable oil and dimethylsulfoxide (DMSO).

The compositions of the invention may be administered orally, topically, parenterally, by inhalation or spray or rectally in dosage unit formulations containing conventional non-toxic pharmaceutically acceptable carriers, adjuvants and vehicles. It is further understood that the best method of administration may be a combination of methods. Oral administration in the form of a pill, capsule, soft gel capsule, elixir, syrup, lozenge, troche, or the like is particularly preferred. The term parenteral as used herein includes subcutaneous injections, intradermal, intravascular (e.g., intravenous), intramuscular, spinal, intrathecal injection or like injection or infusion techniques.

The formulations are preferably in a form suitable for oral use, for example, as tablets, troches, lozenges, aqueous or oily suspensions, dispersible powders or granules, emulsion, hard or soft capsules, soft gel capsules, or syrups or elixirs.

The formulations described herein may be prepared according to any method known in the art for the manufacture of pharmaceutical formulations and nutriceuticals, and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; and dispersing or wetting agents, which may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Formulations of the invention may also be in the form of oil-in-water emulsions and water-in-oil emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth; naturally-occurring phosphatides, for example soy bean, lecithin, and esters or partial esters derived from fatty acids and hexitol; anhydrides, for example sorbitan monooleate; and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The formulations may be in the form of a patch, sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

For administration to non-human animals, the formulations of the invention may be added to the animal's feed or drinking water. Also, it will be convenient to formulate animal feed and drinking water products so that the animal takes in an appropriate quantity of the compound in its diet. It will further be convenient to present the compound in a composition as a premix for addition to the feed or drinking water. The composition can also be added as a food or drink supplement for humans.

Dosage levels of the order of from about 5 mg to about 250 mg per kilogram of body weight per day and more preferably from about 25 mg to about 150 mg per kilogram of body weight per day, are useful in the treatment of the above-indicated conditions. The amount of active ingredient that may be combined with the carrier materials to produce a single dosage form will vary depending upon the condition being treated and the particular mode of administration. Dosage unit forms will generally contain between from about 1 mg to about 500 mg of an active ingredient.

Frequency of dosage may also vary depending on the compound used and the particular disease treated. However, for treatment of most disorders, a dosage regimen of 4 times daily or less is preferred. It will be understood, however, that the specific dose level for any particular patient will depend upon a variety of factors including the activity of the specific compound employed, the age, body weight, general health, sex, diet, time of administration, route of administration and rate of excretion, drug combination and the severity of the particular disease undergoing therapy.

The present invention also provides packaged formulations of the invention and instructions for use of the tablet, capsule, soft gel capsule, elixir, etc. Typically, the packaged formulation, in whatever form, is administered to an individual in need thereof that requires an increase in the amount of ubiquinone/ubiquinol in the individual's diet. Typically, the dosage requirement is between about 1 to about 4 dosages a day.

In another aspect, the formulation comprises (a) a ubiquinone/ubiquinol; (b) a surfactant which is a member selected from PTS, PSS, PCS, and PQS. In an exemplary embodiment, the formulation further comprises gelatin. In an exemplary embodiment, the formulation further comprises sorbitol. In an exemplary embodiment, the formulation further comprises glycerin. In an exemplary embodiment, the formulation further comprises purified water. In an exemplary embodiment, the formulation further comprises polysorbate 80. In an exemplary embodiment, the formulation further comprises hydroxylated lechitin. In an exemplary embodiment, the formulation further comprises medium chain triglycerides. In an exemplary embodiment, the formulation further comprises annato seed extract. In an exemplary embodiment, the formulation further comprises soybean oil. In an exemplary embodiment, the formulation further comprises omega-3 enriched fish oil.

In another aspect, the formulation comprises (a) a ubiquinone/ubiquinol; (b) a surfactant which is a member selected from PTS, PSS, PCS, and PQS; gelatin; sorbitol; glycerin; purified water; polysorbate 80; hydroxylated lechitin; medium chain triglycerides, annato seed extract and soybean oil. This formulation can optionally further include a vitamin and/or preservative, such as vitamin E. In another exemplary embodiment, the formulation comprises (a) a ubiquinone/ubiquinol; (b) PTS; gelatin; sorbitol; glycerin; purified water; polysorbate 80; hydroxylated lechitin; medium chain triglycerides, annato seed extract, and omega-3 enriched fish oil.

In another aspect, the formulation comprises (a) a ubiquinone/ubiquinol; (b) a surfactant which is a member selected from PTS, PSS, PCS, and PQS. In an exemplary embodiment, the formulation further comprises titanium dioxide. In an exemplary embodiment, the formulation further comprises riboflavin.

In another aspect, the formulation comprises (a) a ubiquinone/ubiquinol; (b) a surfactant which is a member selected from PTS, PSS, PCS, and PQS; soybean oil; gelatin; glycerin; beeswax; lechitin; titanium dioxide; and riboflavin. In another exemplary embodiment, the formulation comprises (a) a ubiquinone/ubiquinol; (b) PTS; gelatin; sorbitol; glycerin; purified water; polysorbate 80; hydroxylated lechitin; medium chain triglycerides, annato seed extract, and omega-3 enriched fish oil.

In another aspect, the formulation comprises (a) a ubiquinone/ubiquinol; (b) a surfactant which is a member selected from PTS, PSS, PCS, and PQS. In an exemplary embodiment, the formulation further comprises rice bran oil. In an exemplary embodiment, the formulation further comprises beeswax. In an exemplary embodiment, the formulation further comprises carotenoids.

In another aspect, the formulation comprises (a) a ubiquinone/ubiquinol; (b) a surfactant which is a member selected from PTS, PSS, PCS, and PQS; rice bran oil; beeswax; and carotenoids. In another exemplary embodiment, the formulation comprises (a)

### VI. Methods of Making the Formulations

In another aspect, the invention provides a method of making the formulations described herein.

In an exemplary embodiment, the method of making the formulation comprises (i) contacting said ubiquinone/ubiquinol and said surfactant; and (ii) contacting the product of step (i) with said lipophilic carrier and said viscosity enhancer, thereby making the formulation.

In another exemplary embodiment, the method of making the formulation comprises (i) melting the beeswax in the lipophilic carrier and heating the resulting mixture to a minimum of 50° C. until the wax has melted completely and the solution is clear. The method may further include any of the following steps: (ii) cooling the mixture to at least 30° C.; (iii) adding the viscosity enhancer; (iv) mixing the intermediate mixture for at least 20 minutes; (v) adding the ubiquinone/ubiquinol, preferably at a temperature of 28° C. or less; and mixing the product of step (v) for a minimum of 30 minutes to form the formulation.

In another exemplary embodiment, the method of making the formulation comprises: (i) heating the lipophilic carrier to 50 to 60° C. (ii) adding beeswax (50° C. is above the melting point of bees wax); (iii) mixing the wax and lipophilic carrier until a uniform mixture is formed. Bees wax thickens the lipophilic carrier and acts as a suspension agent for subsequent ingredients. Without bees wax, the other ingredients, when suspended inside a transparent gel capsule, might separate or congregate under the effect of gravity, and appear faulty or spoiled to the consumer. The method may further include (iv) cooling the mixture of step (iii) to 35 to 45° C.; (v) adding ubiquinone/ubiquinol under a vacuum (to eliminate oxidation) and mixing the resulting mixture for one to two hours; (vi) cooling the resultant mixture to 25 to 30° C. A nitrogen gas blanket is introduced to shield the mixture for oxygen and the pressure is returned to atmospheric. The mixture is then encapsulated in a soft gel capsule.

In another exemplary embodiment, the method of making the formulation comprises: (i) Mixing all ingredients under a nitrogen blanket and maintain this blanket throughout blending; (ii) Melting the viscosity enhancer as well as other components in the lipophilic carrier, and heating the mixture to a minimum of 60° C.; (iii) Allowing the mixture to cool to at least 26° C. and adding CoQ₁₀; (iv) Mixing the solution for a minimum of 30 minutes to assure the mixture is homogenous and that no air remains; and (v) Encapsulate in a gel capsule.

### IV. Non-(Ubiquinone and/or Ubiquinol) Formulations

The current invention provides novel formulations with improved bioavailability for ingredients other than ubiquinone and/or ubiquinol (e.g., CoQ₁₀). This first ingredient is a member selected from asiatic acid, ursolic acid, lutein, astaxanthin, curcumins, beta-carotene, lycopene, resveratrol, lecithin, L-carnitine (or acetyl derivative), tocotrienols, alpha-lipoic acid, salmon oil, grape seed extract, bilberry extract, flaxseed oil, garlic oil, ginkgo biloba extract, pumpkin seed oil, green tea catechins extract, kava, evening primrose oil, wheat germ oil, hyaluronic acid, saw palmetto berry oil extract, ginseng, Japanese knotwood extract, phytosterols, hawthorne, St. John's wort, melatonin, valerian, yohimbe, ephedra, red clover, cayenne, echinacea, arnica Montana, docosahexaenoic acid, analogs thereof (such as ester derivatives and/or amide derivatives) and combinations thereof. First ingredient analogs can include any first ingredient which has had at least one free OH or COOH group converted into an ester. First ingredient analogs can include any first ingredient which has had at least one free NH group converted into an amide. When added to an aqueous solution, the formulations allow for the formation of micelles, wherein particle size of the micelles is surprisingly small, enabling greater bioavailability. An added advantage of the current formulations is their greater health benefits due to the presence of essential polyunsaturated fatty acids, such as omega-3 fatty acids. In addition, the current formulations are stable if they contain a viscosity enhancer (e.g., bees wax) and/or surfactants, relative to the first ingredient, than known formulations. Another advantage of the current formulations is their stability with respect to precipitation of one or more components and the first ingredient in particular. In an exemplary embodiment, non-CoQ₁₀ formulations of the invention are stable under an inert atmosphere or within a soft gel capsule at room temperature for extended amounts of time, such as from about 2 months or more. These formulations can be produced according to a method described herein.

In one aspect, the invention provides a formulation which comprises: (a) a first ingredient which is a member selected from an asiatic acid, ursolic acid, lutein, astaxanthin, curcumins, beta-carotene, lycopene, resveratrol, lecithin, L-carnitine (or acetyl derivative), tocotrienols, alpha-lipoic acid, salmon oil, grape seed extract, bilberry extract, flaxseed oil, garlic oil, ginkgo biloba extract, pumpkin seed oil, green tea catechins extract, kava, evening primrose oil, wheat germ oil, hyaluronic acid, saw palmetto berry oil extract, ginseng, Japanese knotwood extract, phytosterols, hawthorne, St. John's wort, melatonin, valerian, yohimbe, ephedra, red clover, cayenne, echinacea, arnica Montana, docosahexaenoic acid, analogs thereof (such as ester derivatives and/or amide derivatives) and combinations thereof; (b) a surfactant described herein; and a (c) a lipophilic carrier described herein. In an exemplary embodiment, this formulation further comprises (d) a viscosity enhancer described herein.

In an exemplary embodiment, the first ingredient is a member selected from an asiatic acid, ursolic acid, curcumins, resveratrol, lecithin, L-carnitine (or acetyl derivative), tocotrienols, alpha-lipoic acid, salmon oil, grape seed extract, bilberry extract, flaxseed oil, garlic oil, ginkgo biloba extract, pumpkin seed oil, green tea catechins extract, kava, evening primrose oil, wheat germ oil, hyaluronic acid, saw palmetto berry oil extract, ginseng, Japanese knotwood extract, phytosterols, hawthorne, St. John's wort, melatonin, valerian, yohimbe, ephedra, red clover, cayenne, echinacea, arnica Montana, docosahexaenoic acid, analogs thereof (such as ester derivatives and/or amide derivatives) and combinations thereof;

In an exemplary embodiment, the first ingredient is an asiatic acid, wherein said asiatic acid is present in an amount of from about 100 mg to about 500 mg. In an exemplary embodiment, the first ingredient is an ursolic acid wherein said ursolic acid is present in an amount of from about 100 mg to about 500 mg. In an exemplary embodiment, the first ingredient is a lutein wherein said lutein is present in an amount of from about 5 mg to about 50 mg. In an exemplary embodiment, the first ingredient is an astaxanthin, wherein said astaxanthin is present in an amount of from about 2 mg to about 20 mg. In an exemplary embodiment, the first ingredient is a curcumin, wherein said curcumin is present in an amount of about 500 mg. In an exemplary embodiment, the first ingredient is a beta-carotene, wherein said beta-carotene is present in an amount of about 25,000 IU. In an exemplary embodiment, the first ingredient is a lycopene, wherein said lycopene is present in an amount of from about 5 mg to about 50 mg. In an exemplary embodiment, the first ingredient is a resveratrol, wherein said resveratrol is present in an amount of from about 10 mg to about 250 mg. In an exemplary embodiment, the first ingredient is a lecithin, wherein said lecithin is present in an amount of about 5 mg. In an exemplary embodiment, the first ingredient is L-carnitine (or acetyl derivative), wherein said L-carnitine (or acetyl derivative) is present in an amount of about 500 mg. In an exemplary embodiment, the first ingredient is a tocotrienol, wherein said tocotrienol is present in an amount of from about 10 mg to about 200 mg. In an exemplary embodiment, the first ingredient is an alpha-lipoic acid, wherein said alpha-lipoic acid is present in an amount of from about 50 mg to about 800 mg. In an exemplary embodiment, the first ingredient is a salmon oil, wherein said salmon oil is present in an amount of from about 100 mg to about 2000 mg. In an exemplary embodiment, the first ingredient is a grape seed extract, wherein said grape seed extract is present in an amount of from about 20 mg to about 300 mg. In an exemplary embodiment, the first ingredient is a bilberry extract, wherein said bilberry extract is present in an amount of from about 10 mg to about 500 mg. In an exemplary embodiment, the first ingredient is a flaxseed oil, wherein said flaxseed oil is present in an amount of from about 100 mg to about 2000 mg. In an exemplary embodiment, the first ingredient is a garlic oil, wherein said garlic oil is present in an amount of about 5 mg. In an exemplary embodiment, the first ingredient is a ginkgo biloba extract, wherein said ginkgo biloba extract is present in an amount of from about 50 mg to about 500 mg. In an exemplary embodiment, the first ingredient is a pumpkin seed oil, wherein said pumpkin seed oil is present in an amount of from about 100 mg to about 2000 mg. In an exemplary embodiment, the first ingredient is a green tea catechins extract, wherein said green tea catechins extract is present in an amount of from about 50 mg to about 500 mg. In an exemplary embodiment, the first ingredient is a kava, wherein said kava is present in an amount of about 5 mg. In an exemplary embodiment, the first ingredient is a evening primrose oil, wherein said evening primrose oil is present in an amount of from about 100 mg to about 2000 mg. In an exemplary embodiment, the first ingredient is a wheat germ oil, wherein said wheat germ oil is present in an amount of from about 100 mg to about 1000 mg. In an exemplary embodiment, the first ingredient is a hyaluronic acid, wherein said hyaluronic acid is present in an amount of from about 10 mg to about 500 mg. In an exemplary embodiment, the first ingredient is a saw palmetto berry oil extract, wherein said saw palmetto berry oil extract is present in an amount of from about 50 mg to about 500 mg. In an exemplary embodiment, the first ingredient is a ginseng, wherein said ginseng is present in an amount of from about 100 mg to about 1000 mg. In an exemplary embodiment, the first ingredient is a Japanese knotwood extract, wherein said Japanese knotwood extract is present in an amount of from about 10 mg to about 500 mg. In an exemplary embodiment, the first ingredient is a phytosterol, wherein said phytosterol is present in an amount of from about 50 mg to about 1000 mg. In an exemplary embodiment, the first ingredient is hawthorne, wherein said hawthorne is present in an amount of from about 50 mg to about 1000 mg. In an exemplary embodiment, the first ingredient is St. John's wort, wherein said St. John's wort is present in an amount of from about 50 mg to about 1000 mg. In an exemplary embodiment, the first ingredient is melatonin, wherein said melatonin is present in an amount of from about 50 mg to about 1000 mg. In an exemplary embodiment, the first ingredient is valerian, wherein said valerian is present in an amount of from about 50 mg to about 1000 mg. In an exemplary embodiment, the first ingredient is yohimbe, wherein said yohimbe is present in an amount of from about 50 mg to about 1000 mg. In an exemplary embodiment, the first ingredient is ephedra, wherein said ephedra is present in an amount of from about 50 mg to about 1000 mg. In an exemplary embodiment, the first ingredient is red clover, wherein said red clover is present in an amount of from about 100 mg to about 1000 mg. In an exemplary embodiment, the first ingredient is cayenne, wherein said cayenne is present in an amount of from about 100 mg to about 1000 mg. In an exemplary embodiment, the first ingredient is echinacea, wherein said echinacea is present in an amount of from about 100 mg to about 1000 mg. In an exemplary embodiment, the first ingredient is arnica Montana, wherein said arnica Montana is present in an amount of from about 100 mg to about 1000 mg. In an exemplary embodiment, the first ingredient is docosahexaenoic acid, wherein said docosahexaenoic acid is present in an amount of from about 50 mg to about 1000 mg.

In another exemplary embodiment, the formulation is mixed in water. This formulation is a member selected from: (a) a first ingredient which is a member selected from lecithin, garlic oil, kava, and combinations thereof; (b) a surfactant described herein; and a (c) a lipophilic carrier described herein. In an exemplary embodiment, this formulation further comprises (d) a viscosity enhancer described herein. In another exemplary embodiment, about 5 g of the first ingredient first ingredient is present in the aqueous formulation.

### Example 1

### Formulation of Lipophilic Bioactive Molecule Stabilized with Vitamin C

0.700 kg ubiquinone, 2.100 kg PTS-600 and 5.950 kg water were added to a reactor, and the batch was warmed to 90-95° C. (90° C. preferred). The batch was stirred for at least about 1 h at 90-95° C. The batch was cooled to 10-15° C. (10° C. preferred) at a rate greater than 10° C./h using an ice bath. Once the mixture was clear, the reaction was sampled and analyzed. Clarity was determined by a turbidity meter. 5.190 kg water was added. Once the mixture was homogeneous, the reaction was sampled and analyzed. Clarity was determined by a turbidity meter. 2.061 kg ascorbic acid was added. Then, 1.099 kg 7% sodium bicarbonate was added. The final pH must be checked. Care should be taken to stir the mixture long enough to allow carbon dioxide to fully degas. Final pH was targeted at 3.5. Once the mixture was homogeneous, the reaction was sampled. The batch was warmed to 50-55° C. (50° C. preferred) and stirred for at least about 24 h at 50-55° C., The batch was sampled and analyzed. Conversion of ubiquinone to ubiquinol was determined by USP method for ubiquinone. The reaction was considered completed when less than 1% ubiqunone present.

Adjusting the formulation to pH 3.5 allowed the subsequent heating to 50-55° C. without degradation of the solution. Without the pH adjustment, PTS decomposes and undergoes hydrolysis, and the components of the reaction precipitate from solution. The above example thus demonstrates an advantage in the process of making the present formulations.

The above formulation can be further processed and mixed with a spray drying carrier for use in making the various formulations disclosed herein.

The articles “a,” “an” and “the” as used herein do not exclude a plural number of the referent, unless context clearly dictates otherwise. The conjunction “or” is not mutually exclusive, unless context clearly dictates otherwise.

All references, publications, patent applications, issued patents, accession records, databases, websites and document urls cited herein, including those in any appendices and attachments, are incorporated by reference in their entirety for all purposes.

What is claimed is:

1. A formulation comprising:
(a) a lipophilic bioactive molecule;
(b) a spray drying carrier; and
(c) a solubilizing agent having a structure according to:
Y¹L¹ₐZ
wherein a is an integer selected from 0 and 1;
Z is a hydrophobic moiety;
Y¹ is a linear or branched hydrophilic moiety including at least one polymeric moiety; and
L¹ is a linker moiety that covalently links the hydrophobic moiety Z and the hydrophilic moiety Y¹.

2. The formulation of claim 1 wherein said lipophilic bioactive molecule is a member selected from a ubiquinone, ubiquinol, carotenoid, xanthophyll, triterpenoid, pentacyclic triterpenoid, phytosterol, stilbenoid (resveratrol), an essential fatty acid, an oil comprising an omega-3 fatty acid, an oil comprising an omega-6 fatty acid, an oil comprising an omega-9 fatty acid and an oil comprising an omega-12 fatty acid.

3. The formulation of claim 2 wherein said lipophilic bioactive molecule is an omega-3 fatty acid.

4. The formulation of claim 2 wherein said lipophilic bioactive molecule is ubiquinone or ubiquinol.

5. The formulation of any preceding claim wherein said solubilizing agent is polyoxyethanyl tocopheryl sebacate (PTS).

6. The formulation of any preceding claim wherein said spray drying carrier comprises a gum and maltodextrin.

7. The formulation of any preceding claim further comprising a compound selected from the group consisting of a pharmaceutical drug, a sterol, a vitamin, a provitamin, an amino acid, an amino acid analog, a fat, a phospholipid, a carotenoid, a sugar, a starch, an antibiotic, a stabilizer, a reducing agent and a free radical scavenger.

8. The formulation of any preceding claim wherein the weight ratio of said lipophilic bioactive molecule to said solubilizing agent is selected from the group consisting of about 1:2 and about 1:3.

9. The formulation of any preceding claim wherein the weight percent of said bioactive lipophilic molecule is about 1% to about 10%.

10. The formulation of any preceding claim further comprising water.

11. The formulation of claim 10 wherein said formulation is clear.

12. The formulation of claim 11 wherein said formulation is clear at room temperature.

13. The formulation of any of claims 10 to 12 wherein said formulation is colorless.

14. A solid, water-soluble formulation prepared by spray drying the formulation of any of claims 10 to 13.

15. A method of making the formulation of any of claims 1 to 9 comprising spray drying a solution comprising said formulation.
